(19) United States
(12) Patent Application Publication
(10) Pub. No.: US 2015/0210768 A1
(43) Pub. Date: Jul. 30, 2015

(54) Methods and Compounds for Preventing, Treating and Diagnosing an Inflammatory Condition

(71) Applicant: Westfaelische Wilhelms-Universitaet Muenster, Muenster (DE)

(72) Inventors: Johannes Roth, Muenster (DE); Thomas Vogl, Muenster (DE)

(21) Appl. No.: 14/423,751

(22) PCT Filed: Sep. 10, 2013
(86) PCT No.: PCT/EP13/68757

§ 371 Date: Feb. 25, 2015

(30) Foreign Application Priority Data
Sep. 10, 2012 (EP) 12183736.3

(51) Int. Cl. C07K 16/28; C07K 7/08

(52) U.S. Cl. CPC C07K 16/2896; C07K 7/08; C07K 2317/34

(57) ABSTRACT

Provided is an antibody with a specificity to an epitope that is a region corresponding to amino acid positions 63-79 or 73-85 of the human protein S100A9. Provided is further an antibody with a specificity to an epitope that is a region corresponding to amino acid positions 55-71 of the human protein S100A8. Provided is further the use of such antibody in the treatment or diagnosis of an inflammatory disorder. Also provided is an in-vitro method of identifying a compound capable of inhibiting the formation of a complex between a peptide corresponding to one of the above epitopes of S100A9 or the above epitope of S100A8 and a TLR4 receptor, where a compound suspected to affect the complex formation is contacted with the peptide and the TLR4 receptor. Further provided is an in-vitro method of identifying a compound capable of increasing the stability of a complex between a S100A8 protein and a S100A9 protein, where the two proteins are contacted in the presence of a compound suspected to affect the complex formation.

### CROSS-REFERENCE TO RELATED APPLICATIONS

The present application claims the benefit of and the priority to an application for “Methods And Compounds For Preventing, Treating And Diagnosing An Inflammatory Condition” filed on 17 Oct. 2011 with the European Patent Office, and there duly assigned serial number EP 12 183 736. The content of said application filed on 17 Oct. 2011 is incorporated herein by reference for all purposes in their entirety including all tables, figures, and claims—as well as including an incorporation of any element or part of the description, claims or drawings not contained herein and referred to in Rule 20.5(a) of the PCT, pursuant to Rule 4.18 of the PCT.

### FIELD OF THE INVENTION

The present invention relates to methods and compounds for preventing, treating and diagnosing inflammatory conditions in a subject. Provided are further methods of identifying compounds suitable for preventing, treating and diagnosing inflammatory conditions in a subject.

### BACKGROUND OF THE INVENTION

The following discussion of the background of the invention is merely provided to aid the reader in understanding the invention and is not admitted to describe or constitute prior art to the present invention.

Uncontrolled inflammatory processes play an important role in many diseases such as infections, sepsis, septic shock, allergies and auto immune diseases, as well as chronic diseases such as arteriosclerosis. Beside the specific, adaptive immune system unspecific, inflammatory processes of the innate immune system have also been the focus of attention recently. The innate immune system represents the first line of defence against invading pathogens and other external harmful agents. The recognition of conserved structures of various pathogens by specific “Pattern Recognition Receptors” (PRR) is well characterized. PRR include inter alia the family of Toll-like-receptors (TLR), which initiate the activation of the inflammation process, also known as the “Pathogen Associated Molecular Pattern” (PAMP). As an example, during an infection with gram negative bacteria Lipopolysaccharid (LPS) very effectively induces an inflammatory response via the LPS-receptor complex (TLR4/MD2/CD14) in phagocytes, inter alia the induction of proinflammatory cytokines such as TNFα and IL1β.

Therapeutic approaches of blocking TLR4 are already being examined in clinical studies. Furthermore during the last years so-called “Damage Associated Molecular Pattern” (DAMP) have been identified, which are proteins that are being released by activated or necrotic cells during cell stress. These endogenous ligands or “Alarmins” likewise activate PRR, thereby amplifying the inflammatory immune response and enhancing inflammatory reactions. Two DAMP proteins are members of the S100-protein family, namely S100A8 and S100A9.

Current therapies aimed at blocking TLR4—as far as they concern the binding site for endotoxins of gram negative bacteria—encompass an increased risk of infection, since such a therapy inevitably likewise blocks the response to such bacterial products. It would thus be desirable to be able to inhibit inflammation reactions by an approach that avoids this adverse effect.

### SUMMARY OF THE INVENTION

Provided herein are methods and compounds that are suitable for inhibiting inflammation reactions in a vertebrate organism. In contrast to conventional therapeutic approaches a method or use as described herein involves affecting the action of two endogenous TLR4 ligands, namely S100A8/S100A9. Thereby such a use or method is substantially more specific than conventional approaches.

In blood of healthy individuals the proteins S100A8 and S100A9 are present in the form of an inactive complex. For their pro-inflammatory function to unfold, the proteins need to be activated. The present inventors have identified this activation mechanism, and thereby also a very specific starting point for novel approaches of anti-inflammatory therapies.

In a first aspect the present invention provides a compound that has a binding specificity to an epitope of a vertebrate S100A9 protein. The epitope has an amino acid sequence of a region, which corresponds to the amino acid that spans the range from amino acid position 63 to amino acid position 79 of the human protein S100A9 of the Uniprot/Swissprot accession number P06702 (version 147 as of 5 Sep. 2012, SEQ ID NO: 77). Any reference to “the” human protein S100A9 concerns the protein of the sequence of this data base entry. This region, i.e. amino acid positions 63-79 of the human protein S100A9, also corresponds to the amino acid sequence that spans the range from amino acid position 63 to amino acid position 79 of the bovine protein S100A9. This region also corresponds to the amino acid sequence from amino acid position 62 to amino acid position 78 of the putative horse protein S100A9 (Swissprot/Uniprot accession No F6RM82, version 10 of 5 Sep. 2012, SEQ ID NO: 79). The region also corresponds to the amino acid sequence from amino acid position 62 to amino acid position 78 of the putative marmoset protein S100A9 (Swissprot/Uniprot accession no F7ID42, version 8 as of 5 Sep. 2012, SEQ ID NO: 80). The region also corresponds to the amino acid sequence from amino acid position 62 to amino acid position 78 of the putative marmoset protein S100A9 (Swissprot/Uniprot accession No. F7ID42, version 15 of 24 Jul. 2013, SEQ ID NO: 81). As a further example, this region corresponds to the amino acid sequence from amino acid position 63 to amino acid position 79 of the bovine protein S100A9 (Swissprot/Uniprot accession No E1BLI9, version 14 of 29 May 2013, SEQ ID NO: 85). In typical embodiments the compound according to the first aspect is an immunoglobulin or a proteinaceous binding partner with a binding specificity to the above epitope.

A vertebrate S100A9 protein is understood to include any naturally occurring variant of a vertebrate S100A9 protein. In some embodiments the compound according to the first aspect is a compound for use as a medicament or for use in diagnosis.

In a second aspect the present invention provides a compound that has a binding specificity to an epitope of a vertebrate S100A9 protein. The epitope has an amino acid sequence of a region that corresponds to the amino acid sequence that spans the range from amino acid position 73 to amino acid position 85 of the human protein S100A9 of SEQ ID NO: 77 (cf. below). This region also corresponds to the amino acid sequence from amino acid position 72 to amino acid position 84 of the putative horse protein S100A9 (Swissprot/Uniprot accession No F6RM82, version 10 of 5 Sep. 2012, SEQ ID NO: 79). In typical embodiments the compound according to the second aspect is an immunoglobulin or a proteinaceous binding partner with a binding specificity to the above epitope.

In some embodiments the compound according to the second aspect is a compound for use as a medicament or for use in diagnosis.

In a third aspect the present invention provides a compound that has a binding specificity to an epitope of a vertebrate S100A8 protein. The epitope has an amino acid sequence of a region that corresponds to the amino acid sequence that spans the range from amino acid position 55 to amino acid position 71 of the human protein S100A8, which has Uniprot/Swissprot accession number P05109 (version 138 as of 5 Sep. 2012, SEQ ID NO: 78). Any reference to “the” human protein S100A8 concerns the protein of the sequence of this data base entry. This region, i.e. amino acid positions 55-71 of the human protein S100A8, also corresponds to the amino acid sequence from amino acid position 58 to amino acid position 73 of the putative opossum protein S100A8 (Swissprot/Uniprot accession No F6SK92, version 9 of 5 Sep. 2012, SEQ ID NO: 82). In typical embodiments the compound according to the third aspect is an immunoglobulin or a proteinaceous binding partner with a binding specificity to the above epitope.

A vertebrate S100A8 protein is understood to include to any naturally occurring variant of a vertebrate S100A8 protein. In some embodiments the compound according to the third aspect is a compound for use as a medicament or for use in diagnosis.

In a fourth aspect the present invention provides a combination of a compound according to the first aspect and a compound according to the third aspect. In some embodiments the combination further includes a compound according to the second aspect. In some embodiments the combination according to the fourth aspect is included in a single compound, such as a single immunoglobulin or proteinaceous binding partner. Such an immunoglobulin or proteinaceous binding partner typically has at least a dual binding specificity.

In some embodiments the combination according to the fourth aspect is a combination for use as a medicament or for use in diagnosis.

In a fifth aspect the present invention provides a combination of a compound according to the second aspect and a compound according to the third aspect. In some embodiments the combination according to the fifth aspect is included in a single compound, such as a single immunoglobulin or proteinaceous binding partner. Such an immunoglobulin or proteinaceous binding partner typically has at least a dual binding specificity.

In some embodiments the combination according to the fifth aspect is a combination for use as a medicament or for use in diagnosis.

In a sixth aspect the present invention provides a combination of a compound according to the first aspect and a compound according to the second aspect. In some embodiments the combination according to the sixth aspect is included in a single compound, such as a single immunoglobulin or proteinaceous binding partner. Such an immunoglobulin or proteinaceous binding partner typically has at least a dual binding specificity.

In some embodiments the combination according to the sixth aspect is a combination for use as a medicament or for use in diagnosis.

In a seventh aspect the present invention provides a method of treating a subject suffering from an inflammatory disorder. The method includes administering to the subject a compound according to the first aspect and/or a compound according to the second aspect.

In an eighth aspect the present invention provides a method of treating a subject suffering from an inflammatory disorder. The method includes administering to the subject a compound according to the third aspect.

In a ninth aspect the present invention provides a method of treating a subject suffering from an inflammatory disorder. The method includes administering to the subject a combination according to the fourth, fifth or sixth aspect.

In a tenth aspect the present invention provides an isolated peptide or peptidomimetic. The peptide or peptidomimetic includes, essentially consists of, or consists of the sequence of X₃EX₂X₃X₁X₁X₁X₁X₁X₁ X₅X₁X₁X₆X₂X₁X₁ (SEQ ID NO: 6). X₁ in this sequence and any other sequence disclosed in this document represents any amino acid. X₂ in this sequence and any other sequence disclosed in this document represents an amino acid with a side chain that carries a carboxylic acid group. X₃ in this sequence and any other sequence disclosed in this document represents a non-polar amino acid. X₅ in this sequence and any other sequence disclosed in this document represents one of the amino acids D, N, E or Q. X₆ in this sequence and any other sequence disclosed in this document represents an aromatic amino acid.

Generally a peptide according to the tenth aspect differs from a full-length calcium binding protein. In some embodiments a peptidomimetic according to the tenth aspect has a sequence that differs from the sequence of a full-length S100 protein such as S100A9, being the full-length protein Calgranulin-B.

The peptide according to the tenth aspect typically has a length of 150 amino acids or less, such as 120 amino acids or less. In some embodiments the peptide typically has a length of 100 amino acids or less. In some embodiments the peptide typically has a length of 80 amino acids or less. In some embodiments the peptide typically has a length of 60 amino acids or less. In some embodiments the peptide typically has a length of 50 amino acids or less. In some embodiments the peptide typically has a length of 40 amino acids or less. In some embodiments the peptide typically has a length of 30 amino acids or less.

In some embodiments an isolated peptide or peptidomimetic according to the tenth aspect includes, essentially consists of, or consists of the sequence of X₃EX₂X₃X₂X₁X₄ X₁X₅X₁X₅X₁X₁X₆X₂X₂X₁ (SEQ ID NO: 66), or a homolog thereof.

In some embodiments an isolated peptide or peptidomimetic according to the tenth aspect includes, essentially consists of, or consists of the sequence of X₃EX₂X₃X₂X₁X₄X₁X₅X₁QX₁X₆X₁EX₂X₁ (SEQ ID NO: 64), or a homolog thereof X₄ in this sequence and any other sequence disclosed in this document represents one of the amino acids N or Q.

In some embodiments an isolated peptide or peptidomimetic according to the tenth aspect includes, essentially consists of, or consists of the sequence of MEX₂X₁X₁X₁NX₁X₁X₁QX₁X₁FEX₁X₁ (SEQ ID NO: 67), or a homolog thereof.

In some embodiments an isolated peptide or peptidomimetic according to the tenth aspect includes, essentially consists of, or consists of the sequence of MEX₂X₃X₈X₁X₁X₁ X₁X₁QX₁X₁FEX₈X₁ (SEQ ID NO: 74), or a homolog thereof X₈ in this sequence and any other sequence disclosed in this document represents a polar amino acid.

In some embodiments an isolated peptide or peptidomimetic according to the tenth aspect includes, essentially consists of, or consists of the sequence of MEX₂X₃X₈X₁X₈X₁ X₈X₁QX₁X₁FEX₂X₁ (SEQ ID NO: 75), or a homolog thereof.

In some embodiments an isolated peptide or peptidomimetic according to the tenth aspect includes, essentially consists of, or consists of the sequence of MEX₂X₃X₂X₁X₂X₁ X₂X₁QX₁X₁FEX₈X₁ (SEQ ID NO: 76), or a homolog thereof.

In some embodiments an isolated peptide or peptidomimetic according to the tenth aspect includes, essentially consists of, or consists of the sequence of MEX₂X₃DX₁NX₁DX₁QX₁X₁FEX₂X₁ (SEQ ID NO: 7), or a homolog thereof.

In some embodiments an isolated peptide or peptidomimetic according to the tenth aspect includes, essentially consists of, or consists of the sequence of MEDX₃X₁X₃X₁X₁DX₁ QX₃X₁FEX₁X₁ (SEQ ID NO: 72), or a homolog thereof.

In some embodiments an isolated peptide or peptidomimetic according to the tenth aspect includes, essentially consists of, or consists of the sequence of MEDX₃X₂X₃X₅X₁X₅X₁ QX₃X₁FEX₂X₁ (SEQ ID NO: 73), or a homolog thereof.

In some embodiments an isolated peptide or peptidomimetic according to the tenth aspect includes, essentially consists of, or consists of the sequence of MEDX₃DX₃NX₁DX₁ QX₃X₁FEEX₁ (SEQ ID NO: 8), or a homolog thereof.

In some embodiments a peptide or peptidomimetic of the tenth aspect consists of, includes or essentially consists of a homolog of the sequence of SEQ ID NO: 6.

In a eleventh aspect the present invention provides an isolated peptide or peptidomimetic. The peptide or peptidomimetic includes, essentially consists of, or consists of the sequence of X₅X₁X₁X₆X₂X₁X₁ X₁X₃X₃ X₃X₃X₁ (SEQ ID NO: 9). X₁, X₂, X₃, X₅ and X₆ in this sequence are as defined above. Generally a peptide according to the eleventh aspect differs from a calcium binding protein. In some embodiments a peptidomimetic according to the eleventh aspect has a sequence that differs from the sequence of a calcium binding protein.

Generally a peptide according to the eleventh aspect differs from a full-length calcium binding protein. In some embodiments a peptidomimetic according to the eleventh aspect has a sequence that differs from the sequence of a full-length S100 protein such as S100A9, being the full-length protein Calgranulin-B.

The peptide according to the eleventh aspect typically has a length of 150 amino acids or less, such as 120 amino acids or less. In some embodiments the peptide typically has a length of 100 amino acids or less. In some embodiments the peptide typically has a length of 80 amino acids or less. In some embodiments the peptide typically has a length of 60 amino acids or less. In some embodiments the peptide typically has a length of 50 amino acids or less. In some embodiments the peptide typically has a length of 40 amino acids or less. In some embodiments the peptide typically has a length of 30 amino acids or less.

In some embodiments an isolated peptide or peptidomimetic according to the eleventh aspect includes, essentially consists of, or consists of the sequence of X₅X₁X₁X₆X₂X₂X₁ X₁X₃X₃X₃X₃X₁ (SEQ ID NO: 68), or a homolog thereof.

In some embodiments an isolated peptide or peptidomimetic according to the eleventh aspect includes, essentially consists of, or consists of the sequence of QX₁X₁FEX₂X₁X₁X₃X₃X₃X₃X₇ (SEQ ID NO: 10), or a homolog thereof. X₇ in this sequence and any other sequence disclosed in this document represents one of the amino acids R or K.

In some embodiments an isolated peptide or peptidomimetic according to the eleventh aspect includes, essentially consists of, or consists of the sequence of QX₁X₆X₁EX₂X₁X₁X₃X₃X₃X₃X₇ (SEQ ID NO: 65), or a homolog thereof.

In some embodiments an isolated peptide or peptidomimetic according to the eleventh aspect includes, essentially consists of, or consists of the sequence of QX₃X₁FEEX₁X₁ML MX₃X₇ (SEQ ID NO: 11), or a homolog thereof. In some embodiments a peptide or peptidomimetic of the eleventh aspect consists of, includes or essentially consists of a homolog of the sequence of SEQ ID NO: 6.

In a twelfth aspect the present invention provides an isolated peptide or peptidomimetic. The peptide or peptidomimetic includes, essentially consists of, or consists of the sequence of X₆X₈X₅X₃X₁X₁X₁X₁X₁X₁ X₁X₁NX₃X₅X₁X₆ (SEQ ID NO: 12), or a homolog of this sequence. X₁, X₂, X₃, X₅ and X₆ in this sequence are as defined above. X₅ represents D, N, E or Q. X₈ in this sequence and any other sequence disclosed in this document represents a polar amino acid. Generally a peptide or peptidomimetic according to the twelfth aspect differs from a calcium binding protein.

In some embodiments an isolated peptide or peptidomimetic according to the twelfth aspect includes, essentially consists of, or consists of the sequence of FX₈X₅X₃X₁X₁X₁X₁X₁X₁X₁X₁NX₃X₅X₁F (SEQ ID NO: 2), or a homolog thereof.

In some embodiments an isolated peptide or peptidomimetic according to the twelfth aspect includes, essentially consists of, or consists of the sequence of FX₈X₅X₃X₁X₁X₈X₁X₁X₁X₁X₁NX₃X₅X₁F (SEQ ID NO: 4), or a homolog thereof.

In some embodiments an isolated peptide or peptidomimetic according to the twelfth aspect includes, essentially consists of, or consists of the sequence of FX₈X₅X₃X₂X₁X₈X₁DX₁X₁X₁NX₃X₅X₁F (SEQ ID NO: 69), or a homolog thereof.

In some embodiments an isolated peptide or peptidomimetic according to the twelfth aspect includes, essentially consists of, or consists of the sequence of FX₈X₅X₃X₂X₁X₈X₁X₁X₁X₁X₁NX₃X₅EF (SEQ ID NO: 70), or a homolog thereof.

In some embodiments an isolated peptide or peptidomimetic according to the twelfth aspect includes, essentially consists of, or consists of the sequence of FX₈X₅X₃X₂X₁X₈X₁X₁X₁X₁X₁NX₃X₅EF (SEQ ID NO: 71), or a homolog thereof.

In some embodiments an isolated peptide or peptidomimetic according to the twelfth aspect includes, essentially consists of, or consists of the sequence of FX₈EX₃DX₁NX₁DX₉X₁X₁₀NX₁₁X₅EF (SEQ ID NO: 13), or a homolog thereof. In some embodiments a peptide or peptidomimetic of the twelfth aspect consists of, includes or essentially consists of a homolog of the sequence of SEQ ID NO: 6.

Generally a peptide according to the twelfth aspect differs from a full-length calcium binding protein. In some embodiments a peptide or peptidomimetic according to the twelfth aspect has a sequence that differs from the sequence of a full-length S100 protein such as S100A8. In some embodiments a peptide or peptidomimetic according to the twelfth aspect has a sequence that differs from the sequence of a calmodulin protein.

The peptide according to the twelfth aspect typically has a length of 130 amino acids or less, such as 120 amino acids or less. In some embodiments the peptide typically has a length of 100 amino acids or less. In some embodiments the peptide typically has a length of 80 amino acids or less. In some embodiments the peptide typically has a length of 60 amino acids or less. In some embodiments the peptide typically has a length of 50 amino acids or less. In some embodiments the peptide typically has a length of 40 amino acids or less. In some embodiments the peptide typically has a length of 30 amino acids or less.

For a given sequence disclosed herein, any of the embodiments of individual amino acids for selected amino acid positions of the sequence, including groups and/or subgroups of suitable amino acids, such as X₁, X₂, X₃, X₄, X₅, X₆, X₇, X₈, X₉, X₁₀, X₁₁, X₁₂, X₁₃, X₁₄ or X₁₅ included in any sequence may as such be combined with any other amino acid, group and/or subgroup of suitable amino acids in selected positions shown in any other homologous sequence. Thus the individual amino acids at positions in various embodiments of a peptide or peptidomimetic disclosed herein may be combined with each other to provide yet a further embodiment of the respective peptide or peptidomimetic. Where such amino acids, groups or subgroups of amino acids shown as embodiments of a particular sequence correspond to amino acid positions of another sequence, these amino acids, groups or subgroups of amino acids can individually be combined in either sequence with amino acids, groups or subgroups of amino acids shown in the context of any such sequence. The same applies to embodiments of individual amino acids at selected positions denominated by a generic variable such as X₁, X₂, X₃ or X₄, including groups and/or subgroups of suitable amino acids that are shown below, i.e. positions of amino acids or groups/subgroups of amino acids shown as embodiments of a particular sequence. Hence, where a sequence includes for example an amino acid denoted as X₇ and an amino acid denoted as X₅, any of the combinations of as X₇ being R and X₇ being K with any one of D, N, E or Q representing X₅ are within the disclosure of this document. As an illustrative example, the combination of X₇ being R and X₅ being D is equally included as the combination of X₇ being R and X₅ being Q or of X₇ being K and X₅ being D.

In a thirteenth aspect the present invention provides a combination of an isolated peptide or peptidomimetic according to the tenth aspect and an isolated peptide or peptidomimetic according to the twelfth aspect. In some embodiments the combination further includes an isolated peptide or peptidomimetic according to the eleventh aspect. In some embodiments the combination of a peptide or peptidomimetic according to the thirteenth aspect is included in a single peptide or peptidomimetic.

In some embodiments the combination according to the thirteenth aspect is a combination for use as a medicament or for use in diagnosis.

In a fourteenth aspect the present invention provides a combination of an isolated peptide or peptidomimetic according to the eleventh aspect and an isolated peptide or peptidomimetic according to the twelfth aspect. In some embodiments the combination of a peptide or peptidomimetic according to the fourteenth aspect is included in a single peptide or peptidomimetic.

In some embodiments the combination according to the fourteenth aspect is a combination for use as a medicament or for use in diagnosis.

In a fifteenth aspect the present invention provides a combination of an isolated peptide or peptidomimetic according to the tenth aspect and an isolated peptide or peptidomimetic according to the eleventh aspect. In some embodiments the combination of a peptide or peptidomimetic according to the fifteenth aspect is included in a single peptide or peptidomimetic.

In some embodiments the combination according to the fifteenth aspect is a combination for use as a medicament or for use in diagnosis.

As indicated above a peptide or peptidomimetic according to the tenth aspect, a peptide or peptidomimetic according to the eleventh aspect and/or peptide or peptidomimetic according to the twelfth aspect may in some embodiments be included in a common peptide, peptidomimetic or hybrid of a peptide and peptidomimetic. In some embodiments the combination of the thirteenth, fourteenth and/or fifteenth aspect is encompassed in a single peptide or peptidomimetic, or a respective peptide/peptidomimetic hybrid.

In a sixteenth aspect the present invention provides an isolated nucleic acid molecule. The nucleic acid molecule includes a sequence that encodes a peptide with the sequence of SEQ ID NO: 6. Generally the encoded peptide differs from the full-length sequence of a calcium binding protein. The encoded peptide typically differs from a full-length S100 protein such as S100A9, being the full-length protein Calgranulin-B.

The peptide encoded by the nucleic acid molecule of the sixteenth aspect typically has a length of 150 amino acids or less, such as 120 amino acids or less. In some embodiments the encoded peptide has a length of 100 amino acids or less. In some embodiments the encoded peptide has a length of 80 amino acids or less, such as 75 or 70 amino acids. In some embodiments the encoded peptide has a length of 60 amino acids or less. In some embodiments the encoded peptide has a length of 50 amino acids or less, including e.g. 45 amino acids. In some embodiments the encoded peptide has a length of 40 amino acids or less. In some embodiments the encoded peptide has a length of 30 amino acids or less.

In a seventeenth aspect the present invention provides an isolated nucleic acid molecule. The nucleic acid molecule includes a sequence that encodes a peptide with the sequence of SEQ ID NO: 9. Generally the encoded peptide differs from the full-length sequence of a calcium binding protein. The encoded peptide typically differs from a full-length S100 protein such as S100A9, being the full-length protein Calgranulin-B.

The encoded peptide typically has a length of 150 amino acids or less, such as 120 amino acids or less. In some embodiments the encoded peptide has a length of 100 amino acids or less, such as 95, 90 or 85 amino acids. In some embodiments the encoded peptide has a length of 80 amino acids or less. In some embodiments the encoded peptide has a length of 60 amino acids or less. In some embodiments the encoded peptide has a length of 50 amino acids or less. In some embodiments the encoded peptide has a length of 40 amino acids or less. In some embodiments the encoded peptide has a length of 30 amino acids or less.

In an eighteenth aspect the present invention provides an isolated nucleic acid molecule. The nucleic acid molecule includes a sequence that encodes a peptide with the sequence of SEQ ID NO: 12, or a homolog thereof. Generally the encoded peptide differs from the full-length sequence of a calcium binding protein.

Generally the peptide encoded by the nucleic acid molecule according to the eighteenth aspect differs from a full-length calcium binding protein. In some embodiments the encoded peptide has a sequence that differs from the sequence of a full-length S100 protein such as S100A8. In some embodiments the encoded peptide has a sequence that differs from the sequence of a calmodulin protein.

The peptide encoded by the nucleic acid molecule of the eighteenth aspect typically typically has a length of 130 amino acids or less, such as 120 amino acids or less. In some embodiments the peptide has a length of 100 amino acids or less. In some embodiments the peptide has a length of 80 amino acids or less. In some embodiments the peptide has a length of 60 amino acids or less. In some embodiments the peptide has a length of 50 amino acids or less. In some embodiments the peptide typically has a length of 40 amino acids or less, such as 35 amino acids. In some embodiments the peptide typically has a length of 30 amino acids or less.

In an nineteenth aspect the present invention provides an isolated nucleic acid molecule. The nucleic acid molecule includes a combination of a sequence encoding a peptide with the sequence of SEQ ID NO: 6 and a sequence encoding a peptide with the sequence of SEQ ID NO: 12. In some embodiments the nucleic acid molecule according to the nineteenth aspect further includes a sequence encoding a peptide with the sequence of SEQ ID NO: 9.

In a twentieth aspect the present invention provides an isolated nucleic acid molecule. The nucleic acid molecule includes a combination of a sequence that encodes a peptide with the sequence of SEQ ID NO: 9 and a sequence that encodes a peptide with the sequence of SEQ ID NO: 12.

In a twenty-first aspect the present invention provides an isolated nucleic acid molecule. The nucleic acid molecule includes a combination of a sequence encoding a peptide with the sequence of SEQ ID NO: 6 and a sequence that encodes a peptide with the sequence of SEQ ID NO: 9.

In a twenty-second aspect the present invention provides an in-vitro method of identifying a compound, which is capable of decreasing or inhibiting the formation of a complex between a peptide and/or peptidomimetic and a Toll-like receptor 4 (TLR4) protein or a functional fragment of a TLR4 receptor protein. The peptide and/or peptidomimetic includes (i) the amino acid sequence of SEQ ID NO: 6 or 9 and/or (ii) the amino acid sequence of SEQ ID NO: 12. The functional fragment of the TLR4 receptor includes the binding site for SEQ ID NO: 1 and/or for SEQ ID NO: 3, as applicable. The method generally includes providing the peptide and/or peptidomimetic. The method generally also includes providing the TLR4 receptor or the functional fragment of the TLR4 receptor. Furthermore the method generally includes providing a compound suspected to affect the formation of a complex between the peptide and/or peptidomimetic and the TLR4 receptor or the functional fragment of a TLR4 receptor. Further the method includes allowing the peptide and/or peptidomimetic, the TLR4 receptor, or the functional fragment thereof, and the compound to contact each other. The method also includes detecting the formation of a complex between the peptide and/or peptidomimetic and the TLR4 receptor, or the functional fragment of a TLR4 receptor. As indicated above, the peptide and/or peptidomimetic with the sequence of SEQ ID NO: 6 or 9 and the peptide and/or peptidomimetic with the sequence of SEQ ID NO: 12 may in some embodiments be included in a common peptide, peptidomimetic or peptide/peptidomimetic hybrid.

In some embodiments of the method according to the twenty-second aspect the detection is performed by a suitable spectroscopical, photochemical, photometric, fluorometric, radiological, enzymatic or thermodynamic technique.

In some embodiments the method according to the twenty-second aspect includes comparing the formation of the complex to a control measurement. Such a control measurement may for instance include detecting the formation of the complex between the peptide and/or peptidomimetic and a TLR4 protein, or a functional fragment thereof, in the absence of a compound suspected to affect the complex formation.

In a twenty-third aspect the present invention provides an in-vitro method of identifying a compound, which is capable of increasing the stability of a complex between a S100A8 protein, or a functional fragment of a S100A8 protein, and a S100A9 protein, or functional fragment of a S100A9 protein. The method generally includes providing the S100A8 protein, or the functional fragment of a S100A8 protein. The method generally also includes providing the S100A9 protein, or the functional fragment of a S100A9 protein. The method furthermore generally includes providing a compound suspected to affect the formation of a complex between a S100A8 protein, or a functional fragment of a S100A8 protein, and a S100A9 protein or a functional fragment of a S100A9 protein. The method also includes allowing the S100A8 protein, or the functional fragment of a S100A8 protein, the S100A9 protein, or the functional fragment of a S100A9 protein, and the compound that is suspected to affect the complex formation to contact each other. The method further includes detecting the formation of a complex between the S100A8 protein, or the functional fragment of a S100A8 protein, and the S100A9 protein, or the functional fragment of a S100A9 protein.

In some embodiments of the method according to the twenty-third aspect the functional fragment of the S100A8 protein and/or the functional fragment of the S100A9 protein contain at least one of EF hand I and EF hand II. In some embodiments of the method according to the twenty-third aspect the S100A8 protein, or the functional fragment thereof, the S100A9 protein, or the functional fragment thereof, and the compound suspected to affect the complex formation are allowed to contact each other in the presence of a salt of calcium. In some embodiments of the method according to the twenty-third aspect the S100A8 protein, or the functional fragment thereof, the S100A9 protein, or the functional fragment thereof, and the respective compound are allowed to contact each other in the presence of a salt of zinc. In some embodiments of the method according to the twenty-third aspect the S100A8 protein, or the functional fragment thereof, the S100A9 protein, or the functional fragment thereof, and the respective compound are allowed to contact each other in the presence of a salt of copper.

In some embodiments the method according to the twenty-third aspect includes detecting the formation of a heterotetrameric complex between the S100A8 protein, or the functional fragment thereof, and the S100A9 protein, or the functional fragment thereof. The method of such embodiments is a method of identifying a compound capable of increasing the stability of a heterotetrameric complex between a S100A8 protein, or a functional fragment thereof, and a S100A9 protein, or functional fragments thereof.

In some embodiments of the method according to the twenty-third aspect the detection is performed by a suitable spectroscopical, photochemical, photometric, fluorometric, radiological, enzymatic or thermodynamic technique.

In some embodiments the method according to the twenty-third aspect includes comparing the formation of the complex to a control measurement. Such a control measurement may for instance include detecting the formation of the complex between the protein S100A8, or the functional fragment thereof, and the protein S100A9, or the functional fragment thereof, in the absence of a compound suspected to affect the complex formation.

A compound that increases the stability of a complex between a S100A8 protein, or a functional fragment thereof, and a S100A9 protein, or functional fragment thereof, affects the equilibriums existing between the monomeric forms of S100A8 and S100A9, between the heterodimeric complex S100A8/S100A9, and the heterotetrameric complex (S100A8/S100A9)₂. Hence, generally more heterotetrameric complex is formed. As a result, less heterodimeric complex is available, which is capable of binding to the TLR4 receptor.

In some embodiments of a method according to the twenty-third aspect the S100A8 protein, or the functional fragment of a S100A8 protein, the S100A9 protein, or the functional fragment of a S100A9 protein, and the compound suspected to affect the complex formation are allowed to contact each other in the presence of calcium.

In some embodiments a method according to the twenty-third aspect is an in-vitro method of identifying a compound, which is capable of increasing the stability of a heterotetrameric complex between a S100A8 protein, or a functional fragment of a S100A8 protein, and a S100A9 protein, or functional fragment of a S100A9 protein. Typically such a method includes detecting the formation of a heterotetrameric complex between the S100A8 protein, or the functional fragment of a S100A8 protein, and the S100A9 protein, or the functional fragment of a S100A9 protein.

In a twenty-fourth aspect the present invention provides a method of diagnosing the risk of occurrence, or the presence, of a condition associated with an inflammation in a subject. The method includes detecting the amount of a complex between a S100A8 protein and a S100A9 protein in a sample from the subject. A decreased amount of the complex relative to a threshold value indicates an elevated risk of occurrence, or the presence, of a condition associated with an inflammation.

In a twenty-fifth aspect the present invention provides a method of treating a subject suffering from an inflammatory disorder. The method includes administering to the subject a compound obtained by the method of the twenty-third aspect. Administering the compound includes allowing the stability of a complex between a S100A8 protein and a S100A9 protein in a body fluid of the subject to be increased.

In a twenty-sixth aspect the present invention provides a method of treating a subject suffering from an inflammatory disorder. The method includes administering to the subject a compound obtained by the method according to the twenty-second aspect. Administering the compound includes allowing the formation of a complex between the protein S100A8 or the protein S100A9 and a TLR4 receptor on cells of the subject to be decreased or inhibited.

In a twenty-seventh aspect the present invention provides a method of identifying a binding partner of the isolated peptide or peptidomimetic according to the tenth, eleventh and/or twelfth aspect in an organism. The method is generally an in vitro method. The method includes contacting the peptide or peptidomimetic with a sample from the organism. The sample is analysed for the presence of a binding partner of the peptide or peptidomimetic. In some embodiments the sample is also analysed for the identity of a binding partner of the peptide or peptidomimetic. By contacting the peptide or peptidomimetic with the sample a reaction mixture is formed. The method also includes allowing a complex to form between the isolated peptide or peptidomimetic and a binding partner in the reaction mixture. Further the method includes isolating the peptide or peptidomimetic from the reaction mixture. The peptide or peptidomimetic is still present in a complex with the binding partner. The method furthermore includes analysing the binding partner. Analysing the binding partner may include determining one or more physical properties such as its molecular weight. Analysing the binding partner may also include determining whether it is a peptide or protein, a nucleic acid molecule, a lipid, a polysaccharide, a cell a virus or other matter. Where the binding partner is a peptide or protein, a polysaccharide or a nucleic acid molecule, the sequence of the binding partner may further be analysed.

### BRIEF DESCRIPTION OF THE DRAWINGS

FIG. 1: Human monocytes were stimulated for four hours with the indicated concentrations of (A) recombinant human S100A8, recombinant human S100A9 or human S100A8/S100A9, and (B) recombinant human S100A8/S100A9, recombinant human S100A8/S100A9 (N69A) or S100A8/S100A9 (E78A). TNFα released into the culture medium was quantified by means of ELISA.

FIG. 2A shows a section of the 3D structure of the human S100A9 homodimer. The two S100 monomers are shown in shades of grey. Regions that are only accessible in the homodimeric form, but not in the heterodimeric form, are shown in white. Some amino acids are indicated by their position in the human sequence.

FIG. 2B shows a portion of the amino acid sequence of human S100A9. Six amino acids (positions 64, 65, 72, 73, 77 and 85) that are accessible to solvent and that are not involved in calcium coordination or only involved in calcium coordination via their backbone were selected for mutation studies.

FIG. 3A: Tryptic digestion of human S100A9 at indicated points of time. Monocytes were stimulated for four hours with the mixture of fragments, and release of TNFα was quantified via ELISA. The inset depicts a Western Blot for detecting S100A9 that is still intact.

FIG. 3B: Fragments generated by tryptic digestion of human S100A9 were incubated with beads to which TLR4/MD2 was coupled. Fragments bound to the beads were identified via MALDI mass spectrometry. Out of 17 potential peptides only a single peptide could be detected (No. 15: amino acids of positions 73-85) as showing a specific interaction with TLR4/MD2, corresponding to a portion of the C-terminal EF Hand of S100A9.

FIG. 3C shows MALDI mass spectrometry after digestion of a control peptide, as in FIG. 1B. The peptide had the sequence of amino acid positions 63-79 (63-79 5A, molecular weight: 1758 g/mol) of S100A9, in which the four amino acids identified as most likely important for binding to TLR4/MD2 (E64A, D65A, Q73A and E77A, nomenclature of S100A9 maintained), and in addition amino acid K72A, had been exchanged to alanine.

FIG. 3D shows the sequence of the peptide identified. Flanking amino acids are indicated in brackets.

FIG. 3E illustrates schematically the build-up of an immunoprecipitation test of a S100A9 peptide and a S100A8 peptide to TLR4/MD2. 1=agarose bead; 2=peptide; 3=TLR4/MD2.

FIG. 4 depicts the analysis of eluates by MALDI-TOF mass spectrometry. The eluates were obtained following coupling of a peptide, corresponding to positions 63-79 (A) and positions 63-79 AS (B, C), to the TLR4/MD2 complex.

FIG. 5 shows the analysis of eluates by MALDI-TOF mass spectrometry. The eluates were obtained following coupling of a peptide, corresponding to positions 55-71 (A) and 55-71 A3 (B), to the TLR4/MD2 complex.

FIG. 6A illustrates schematically the build-up of a binding test of a S100A9 protein and a S100A9 mutant to TLR4/MD2. FIG. 6B shows the results of an analysis, in which binding of a S100A9 homodimer, or a mutant thereof, to TRLR4/MD2 was detected. The mutants contained an altered amino acid as indicated, i.e. an alanine instead of the naturally occurring amino acid at E64, D65, K72, Q73, E77 or R85. FIG. 6C shows the results of an analysis, in which binding of a S100A9 homodimer, or a mutant thereof, to TRLR4/MD2 was detected. The mutants contained two altered amino acids as indicated, i.e. an alanine instead of the naturally occurring amino acid at both: E64 and D65; Q73 and E77; E64 and Q73; and D65 and Q73.

### DETAILED DESCRIPTION OF THE INVENTION

The present invention can be taken to generally relate to compounds and methods that can be used in the control of inflammatory reactions of an organism. More specifically, compounds and methods are provided for controlling the interaction of an S100A8 protein and/or of an S100A9 protein with a TLR4 receptor.

The protein name “S100” was originally chosen due to the proteins' solubility in 100% ammonium sulphate. S100A8 and S100A9, also known as MRP8 and MRP14, or calgranulin A and calgranulin B, respectively, are two members of the S100 family of Ca²⁺-binding proteins. S100A8 and S100A9 are constitutively expressed in neutrophils, monocytes, and some epithelial cells, while not generally expressed in tissue macrophages or lymphocytes. Monocytes and neutrophil granulocytes express the proteins in large amounts, mainly as S100A8/S100A9 heterodimers. S100A8 and S100A9 proteins contribute to approximately 40-50% of the soluble, cytosolic content of granulocytes. Neutrophils, activated monocytes, and macrophages produce these proteins in response to stress, infection, inflammation, tissue injury, and septic shock. S100A8 and S100A9 are being released at the site of inflammation specifically and in an energy dependent manner, which is tightly controlled. S100A8 and S100A9 are important damage-associated molecular pattern (DAMP) molecules. The S100A8/S100A9 complex is an endogenous ligand of TLR4 on monocytes. Both S100A8 and S100A9 directly bind to the TLR4 receptor complex and induce pro-inflammatory effector mechanisms via the known, classical signal transduction cascade. Hence, S100A8/S100A9 is an important factor in pathogenesis of inflammations.

S100A8 and S100A9 already serve as biochemical markers for chronic and acute inflammation. Both S100 proteins show strong pro-inflammatory activities in many inflammatory reactions, e.g., sepsis, lung and skin infections, arthritis and auto immune diseases. Direct application of S100A8 into the knee joint for instance causes severe joint inflammation and destruction of cartilage. In an experimental mouse model of a T cell dependent autoimmune disease both proteins also induce the generation and activation of autoreactive CD8+ T cells, leading to an increased IL17 mediated immune response.

As calcium-binding cytosolic molecules S100 proteins are characterized by two calcium-binding EF hands with different affinities for calcium connected by a central hinge region. The EF-hand motifs have two α-helices flanking a central calcium-binding loop, thus resulting in a classical helix-loop-helix motif S100A8 and S100A9 can form monovalent homodimers and a heterodimer known as S100A8/A9 (MRP8/14, calprotectin), in the following also referred to as a homodimeric complex and a heterodimeric complex, respectively, as well as even higher oligomeric forms. S100A8 and S100A9 have also been found to form a heterotetramer, in the following also referred to as a heterotetrameric complex. Tetramer formation is strictly dependent on the presence of calcium, and in the absence of calcium, the heterodimer is the preferred form of S100A8 and S100A9.

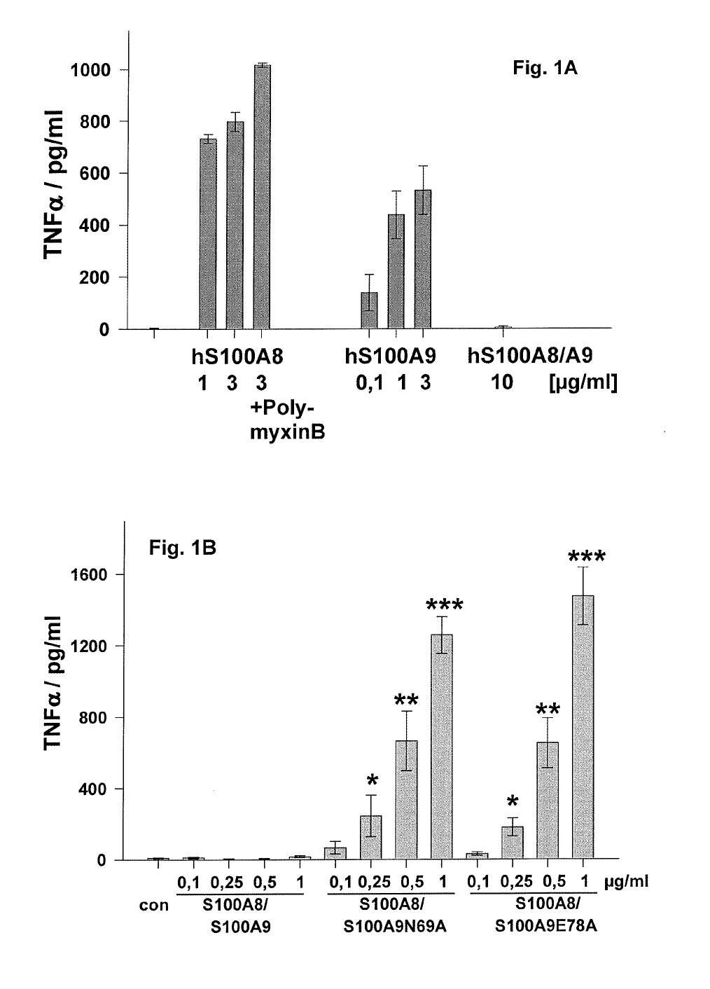

The present invention is based on the identification of a binding site in S100A8 proteins and a binding site in S100A9 proteins for a TLR4 receptor. The invention is further based on the surprising finding that the binding site for a TLR4 receptor, both of S100A8 and of S100A9 proteins, is becoming inaccessible during the formation of a heterotetrameric complex, which is for ease of reference also referred to as (S100A8/S100A8)₂. As can be taken from FIG. 1A, while the heterotetrameric complex between S100A8 and S100A9 does not induce an inflammatory response in monocytes, the individual proteins S100A8 and S100A9 induce a particular strong, pro-inflammatory response in monocytes. This response is comparable to a stimulation by LPS. Likewise homodimers of S100A8 and of S100A9 induce this response.

The Toll-like receptor 4, or TLR4 receptor, also termed CD284, plays an important role in the activation of the innate immune system of an organism, as it detects lipopolysaccharide (LPS), the major component of the outer membrane of Gram-negative bacteria. In some embodiments of a method or a use disclosed herein TLR4 is the human protein with the Swissprot/Uniprot accession No O00206 (version 132 of 5 Sep. 2012). In some embodiments TLR4 is the bovine protein with the Swissprot/Uniprot accession No Q9GL65 (version 88 of 11 Jul. 2012) or with the Swissprot/Uniprot accession No Q8SQ55 (version 56 of 21 Mar. 2012). In some embodiments TLR4 is the rat protein with the Swissprot/Uniprot accession No Q9QX05 (version 99 of 11 Jul. 2012). In some embodiments TLR4 is the mouse protein with the Swissprot/Uniprot accession No Q9QUK6 (version 113 of 5 Sep. 2012). In some embodiments TLR4 is the porcine protein with the Swissprot/Uniprot accession No Q68Y56 (version 62 of 11 Jul. 2012). In some embodiments TLR4 is the chimpanzee protein with the Swissprot/Uniprot accession No H2QXS5 (version 4 of 13 Jun. 2012). In some embodiments TLR4 is the horse protein with the Swissprot/Uniprot accession No F6RL35 (version 10 of 11 Jul. 2012). In some embodiments TLR4 is the chicken protein with the Swissprot/Uniprot accession No C4PCF3 (version 24 of 11 Jul. 2012) or with the Swissprot/Uniprot accession No Q7ZTG5 (version 67 of 5 Sep. 2012). In some embodiments TLR4 is the dog protein with the Swissprot/Uniprot accession No F1PDB9 (version 14 of 5 Sep. 2012).

The present inventors could identify a region on each of S100A8 and S100A9 that is required for the binding of the respective protein to the TLR4 receptor. For the S100A9 protein this sequence corresponds to amino acid positions 63-85 of the human protein (supra). The inventors further found that it is sufficient to prevent the region of the S100A9 protein—for instance by sterically covering it, including by allowing the formation of the heterotetrameric complex described above—which corresponds to amino acid positions 63-79, from binding to a TLR4 receptor. Blocking this region prevents the initiation of the inflammatory response in monocytes. This region also corresponds to amino acid positions 63-79 of the bovine protein, of the gibbon protein, of the Anubis baboon protein, of the bonobo protein, of the panda protein, the porcine protein, the protein of the African elephant or the protein of guinea pig. This region also corresponds to amino acid positions 62-78 of the rat protein encoded by Genbank (NCBI) gene ID: 94195 S100a9, of the mouse protein of NCBI accession No NP_{—}033140.1 (SEQ ID NO: 83) or of the rat protein of NCBI accession No EDM00535.1 (SEQ ID NO: 84). As a further example, this region corresponds to amino acid positions 61-77 of the protein of the Chinese endemic bat species of the mouse-eared bat (David's myotis) of the Swissprot/Uniprot accession No L5MD39 (version 4 of 29 May 2013, SEQ ID NO: 86), or amino acid positions 122-138 of the ferret protein of the Swissprot/Uniprot accession No G9KM87 (version 10 of 24 Jul. 2013, SEQ ID NO: 87).

It is likewise sufficient to prevent the region of the human S100A9 protein corresponding to amino acid positions 73-85 from binding to a TLR4 receptor in order to block the inflammatory response. This region also corresponds to amino acid positions 73-85 of the bovine protein, of the porcine protein, of the protein of the small-eared galago, of the protein of the naked mole rat or the protein of guinea pig.

For the S100A8 protein the inventors have identified the sequence corresponding to amino acid positions 55-71 of the human protein (supra) as necessary for the binding of a S100A8 protein to the TLR4 receptor. This region also corresponds to amino acid positions 55-71 of the macaca protein, of the marmoset protein, of the dog protein, of the protein of the European rabbit, of the ferret protein, of the horse protein, of the bovine protein, of the porcine protein, of the protein of the African elephant, of the panda protein, of the mouse protein, of the rat protein, of the protein of the naked mole rat, of the protein of the Chinese hamster, of the rabbit protein, of the marmoset protein, or of the protein of guinea pig.

The term “position” when used in accordance with this disclosure means the position of either an amino acid within an amino acid sequence depicted herein or the position of a nucleotide within a nucleic acid sequence depicted herein. The term “corresponding” as used herein also includes that a position is not only determined by the number of the preceding nucleotides/amino acids, but is rather to be viewed in the context of the circumjacent portion of the sequence. Accordingly, the position of a given amino acid in accordance with the disclosure which may be substituted may very due to deletion or addition of amino acids elsewhere in a (mutant or wild-type) virus. In this regard it is also noted that data base entries on a nucleic acid sequence of a S100A8 protein or a S100A9 protein may vary in their coverage of non-translated regions, thereby identifying different nucleic acid positions, even though the length of the coding region is unchanged/the same. Similarly, the position of a given nucleotide in accordance with the present disclosure which may be substituted may vary due to deletions or additional nucleotides elsewhere in a non-translated region of a virus, including the promoter and/or any other regulatory sequences or gene (including exons and introns).

Thus, when a position is referred to as a “corresponding position” in accordance with the disclosure it is understood that nucleotides/amino acids may differ in terms of the specified numeral but may still have similar neighbouring nucleotides/amino acids. Such nucleotides/amino acids which may be exchanged, deleted or added are also included in the term “corresponding position”.

Specifically, in order to determine whether an amino acid residue of the amino acid sequence of a S100A8 protein or a S100A9 protein different from a known strain corresponds to a certain position in the amino acid sequence of the known strain, a skilled artisan can use means and methods well-known in the art, e.g., alignments, either manually or by using computer programs such as BLAST2.0, which stands for Basic Local Alignment Search Tool or ClustalW or any other suitable program which is suitable to generate sequence alignments. Accordingly, a known wild-type virus strain may serve as “subject sequence” or “reference sequence”, while the amino acid sequence or nucleic acid sequence of a virus different from the wild-type virus strain described herein can serve as “query sequence”. The terms “reference sequence” and “wild type sequence” are used interchangeably herein.

Provided herein is also a peptide or peptidomimetic, including a peptoid that includes one of the above sequences or a homolog of such a sequence (supra). A homolog is a biologically active sequence that has at least about 70%, including at least about 80% amino acid sequence identity with a given sequence of a polypeptide, such as the sequence of SEQ ID NO: 11. In some embodiments a homolog is a biologically active sequence that has at least about 85% amino acid sequence identity with the native sequence polypeptide. A homolog is a functional equivalent of an isolated nucleic acid molecule or an isolated peptide or protein described in this document. With regard to nucleic acid sequences, the degeneracy of the genetic code permits substitution of certain codons by other codons that specify the same amino acid and hence would give rise to the same protein. The nucleic acid sequence can vary substantially since, with the exception of methionine and tryptophan, the known amino acids can be coded for by more than one codon. Thus, portions or all of the nucleic acid sequences described herein could be synthesized to give a nucleic acid sequence significantly different from that shown in their indicated sequence. The encoded amino acid sequence thereof would, however, be preserved.

In addition, the nucleic acid sequence may include a nucleotide sequence which results from the addition, deletion or substitution of at least one nucleotide to the 5′-end and/or the 3′-end of the nucleic acid formula shown in a given sequence. Any nucleotide or polynucleotide may be used in this regard, provided that its addition, deletion or substitution does not alter the amino acid sequence, which is encoded by the nucleotide sequence. For example, the present invention is intended to include any nucleic acid sequence resulting from the addition of ATG as an initiation codon at the 5′-end of the inventive nucleic acid sequence or its derivative, or from the addition of TTA, TAG or TGA as a termination codon at the 3′-end of the inventive nucleotide sequence or its derivative. Moreover, a nucleic acid molecule may, as necessary, have restriction endonuclease recognition sites added to its 5′-end and/or its 3′-end. Such functional alterations of a given nucleic acid sequence afford an opportunity to promote secretion and/or processing of heterologous proteins encoded by foreign nucleic acid sequences fused thereto.

Further, it is possible to delete codons or to substitute one or more codons with codons other than degenerate codons to produce a structurally modified polypeptide, but one which has substantially the same utility or activity as the polypeptide produced by the unmodified nucleic acid molecule. As recognized in the art, the two polypeptides are functionally equivalent, as are the two nucleic acid molecules that give rise to their production, even though the differences between the nucleic acid molecules are not related to the degeneracy of the genetic code.

“Percent (%) sequence identity” with respect to amino acid sequences disclosed in this document is defined as the percentage of amino acid residues in a candidate sequence that are identical with the amino acid residues in a reference sequence, e.g. of SEQ ID NO: 1, SEQ ID NO: 6, SEQ ID NO: 9 or SEQ ID NO: 12, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publically available computer software such as BLAST, ALIGN, or Megalign (DNASTAR) software. Those skilled in the art can determine appropriate parameters for measuring alignment, including any algorithms needed to achieve maximum alignment over the full length of the sequences being compared. The same is true for nucleotide sequences disclosed herein.

Those skilled in the art will be familiar with the fact that corresponding sequences need to be compared. The use of a corresponding sequence includes that a position is not only determined by the number of the preceding nucleotides/amino acids. Accordingly, the position of a given amino acid in accordance with the disclosure which may be substituted may very due to deletion or addition of amino acids elsewhere in a (mutant or wild-type) protein such as a S100A8 protein or a S100A9 protein. Thus, by a “corresponding position” in accordance with the disclosure it is to be understood that amino acids may differ in the indicated number—for instance when comparing data base entries—but may still have similar neighbouring amino acids (cf. above).

As mentioned above, in some embodiments a sequence such as a sequence corresponding to SEQ ID NO: 11 or SEQ ID NO: 19 contains a conservative substitution. Conservative substitutions are generally the following substitutions, listed according to the amino acid to be mutated, each followed by one or more replacement(s) that can be taken to be conservative: Ala→Gly, Ser, Val; Arg→Lys; Asn→Gln, His; Asp→Glu; Cys→Ser; Gln→Asn; Glu→Asp; Gly→Ala; His→Arg, Asn, Gln; Ile→Leu, Val; Leu→Ile, Val; Lys→Arg, Gln, Glu; Met→Leu, Tyr, Ile; Phe→Met, Leu, Tyr; Ser→Thr; Thr→Ser; Trp→Tyr; Tyr→Trp, Phe; Val→Ile, Leu. Other substitutions are also permissible and can be determined empirically or in accord with other known conservative or non-conservative substitutions. As a further orientation, the following eight groups each contain amino acids that can typically be taken to define conservative substitutions for one another:

1) Alanine (Ala), Glycine (Gly);

2) Aspartic acid (Asp), Glutamic acid (Glu);

3) Asparagine (Asn), Glutamine (Gln);

4) Arginine (Arg), Lysine (Lys);

5) Isoleucine (Ile), Leucine (Leu), Methionine (Met), Valine (Val);

6) Phenylalanine (Phe), Tyrosine (Tyr), Tryptophan (Trp);

7) Serine (Ser), Threonine (Thr); and

8) Cysteine (Cys), Methionine (Met)

In contrast thereto, more substantial changes, such as the following, do not represent conservative substitutions: Ala→Leu, Ile; Arg→Gln; Asn→Asp, Lys, Arg, His; Asp→Asn; Cys→Ala; Gln→Glu; Glu→Gln; His→Lys; Ile→Met, Ala, Phe; Leu→Ala, Met, Norleucine; Lys→Asn; Met→Phe; Phe→Val, Ile, Ala; Trp→Phe; Tyr→Thr, Ser; Val→Met, Phe, Ala.

Sequence alignment and analysis of crystal structures of the S100A8 protein (MRP8) and of the S100A9 protein (MRP14) has previously shown which amino acids are relevant for calcium binding. Ishikawa et al. (Acta Crystallographica Section D [2000] 56, 559-566) for example published the structure of the S100A8 protein. This document is incorporated herein by reference in its entirety. In case of conflict, the present specification, including definitions, will control. By sequence alignment in the sequence FKELDINTDG AVNFQEF of the human protein (SEQ ID NO: 5), which for instance corresponds to the sequence FKELDINKDG AVNFEEF of the porcine protein (SEQ ID NO: 48) or the sequence FKELDINQDN AVNFEEF of the Chinese hamster protein (SEQ ID NO: 53), these authors identified the underlined amino acids as involved in coordinating calcium ions. These amino acids correspond to amino acid positions 5, 7, 9 and 16 of SEQ ID NO: 5. The authors suggested a calcium-triggered conformational change of S100 proteins. Which amino acid residues might be involved in binding to a target protein could, however, not be predicted on the available data.

In the sequence MEDLDTNADK QLSFEEF of the human S100A9 protein (SEQ ID NO: 1), which for instance corresponds to the sequence MEDLDTNVDK QLSFEEF of the bovine protein (SEQ ID NO: 15) or the sequence LEDLDTNADK QLTFEEF of the marmoset protein (SEQ ID NO: 18), these authors identified the underlined amino acids as involved in coordinating calcium ions. These amino acids correspond to amino acid positions 5, 7, 9 and 16 of SEQ ID NO: 1.

In the sequence QLSFEEFIML MAR of the human S100A9 protein (SEQ ID NO: 3) the authors identified the underlined amino acid, corresponding to amino acid position 6 of SEQ ID NO: 3, as involved in coordinating calcium ions.

In uses or methods, in which the formation of a heterotetrameric complex between a S100A8 protein and a S100A9 protein is analysed, the above indicated conserved amino acids should accordingly present, since calcium binding is a requirement for the formation of the heterotetrameric complex. In uses or methods, in which the binding to a TLR4 receptor is analysed, the above indicated conserved amino acids generally need not be present, since binding to the TLR4 receptor occurs only in the homodimeric, heterodimeric or monomeric form of a S100A8 protein or a S100A9 protein.

In some embodiments there is provided an immunoglobulin or a proteinaceous binding partner. The immunoglobulin or proteinaceous binding partner may have a binding specificity to an epitope of a vertebrate S100A9 protein, being an epitope defined by a region that corresponds to amino acid positions 63-79 of the human protein S100A9 and/or a region that corresponds to amino acid position 73-85 of the human protein S100A9. The immunoglobulin or proteinaceous binding partner may also have a binding specificity to an epitope of a vertebrate S100A8 protein, being an epitope defined by a region that corresponds to amino acid positions 55-71 of the human protein S100A8. The terms “specific” and “specificity” as used herein are understood to indicate that the binding partner is directed against, binds to, or reacts with a peptide that has an amino acid sequence of the respective protein region. Thus, being directed to, binding to or reacting with includes that the binding partner specifically binds to a region of a S100A9 protein or of a S100A8 protein, as applicable. The term “specifically” in this context means that the binding partner reacts with the corresponding region of S100A9 or S100A8, as applicable, or/and a portion thereof, but at least essentially not with another protein. The term “another protein” includes any protein, including proteins closely related to or being homologous to e.g. S100A9 and S100A8, against which the binding partner is directed to. The term “does not essentially bind” means that the binding partner does not have particular affinity to another protein, i.e., shows a cross-reactivity of less than about 30%, such as less than about 20%, less than about 10%, including less than about 9, 8, 7, 6 or 5%, when compared to the affinity to S100A9 or S100A8. Whether the binding partner specifically reacts as defined herein above can easily be tested, inter alia, by comparing the reaction of a respective binding partner with S100A9 or S100A8, as applicable, and the reaction of the binding partner with (an) other protein(s). The term “specifically recognizing”, which can be used interchangeably with the terms “directed to” or “reacting with” means in the context of the present disclosure that a particular molecule, generally an immunoglobulin, an immunoglobulin fragment or a proteinaceous binding molecule with immunoglobulin-like functions is capable of specifically interacting with and/or binding to at least two, including at least three, such as at least four or even more amino acids of an epitope as defined herein. Generally the immunoglobulin or proteinaceous binding molecule can thereby form a complex with the respective epitope of S100A9 or S100A8. Such binding may be exemplified by the specificity of a “lock-and-key-principle”. “Specific binding” can also be determined, for example, in accordance with Western blots, ELISA-, RIA-, ECL-, IRMA-tests, FACS, IHC and peptide scans.

A respective binding partner of e.g. S100A9 or S100A8 may be an immunoglobulin, a fragment thereof or a proteinaceous binding partner (i.e. molecule) with immunoglobulin-like functions. Examples of (recombinant) antibody fragments are immunoglobulin fragments such as Fab fragments, Fv fragments, single-chain Fv fragments (scFv), diabodies or domain antibodies (Holt, L. J., et al., Trends Biotechnol. (2003), 21, 11, 484-490). An example of a proteinaceous binding molecule with immunoglobulin-like functions is a mutein based on a polypeptide of the lipocalin family (WO 03/029462, Beste et al., Proc. Natl. Acad. Sci. USA (1999) 96, 1898-1903). Lipocalins, such as the bilin binding protein, the human neutrophil gelatinase-associated lipocalin, human Apolipoprotein D or glycodelin, possess natural ligand-binding sites that can be modified so that they bind to selected small protein regions known as haptens. Examples of other proteinaceous binding molecules are the so-called glubodies (see e.g. international patent application WO 96/23879 or Napolitano, E. W., et al., Chemistry & Biology (1996) 3, 5, 359-367), proteins based on the ankyrin scaffold (Mosavi, L. K., et al., Protein Science (2004) 13, 6, 1435-1448) or crystalline scaffold (e.g. internation patent application WO 01/04144) the proteins described in Skerra, J. Mol. Recognit. (2000) 13, 167-187, AdNectins, tetranectins and avimers. Avimers contain so called A-domains that occur as strings of multiple domains in several cell surface receptors (Silverman, J., et al., Nature Biotechnology (2005) 23, 1556-1561). Adnectins, derived from a domain of human fibronectin, contain three loops that can be engineered for immunoglobulin-like binding to targets (Gill, D. S. & Damle, N. K., Current Opinion in Biotechnology (2006) 17, 653-658). Tetranectins, derived from the respective human homotrimeric protein, likewise contain loop regions in a C-type lectin domain that can be engineered for desired binding (ibid.). Peptoids, which can act as protein ligands, are oligo(N-alkyl)glycines that differ from peptides in that the side chain is connected to the amide nitrogen rather than the a carbon atom. Peptoids are typically resistant to proteases and other modifying enzymes and can have a much higher cell permeability than peptides (see e.g. Kwon, Y.-U., and Kodadek, T., J. Am. Chem. Soc. (2007) 129, 1508-1509). A molecule that forms a complex with a binding partner of S100A9 or S100A8 may likewise be an immunoglobulin, a fragment thereof or a proteinaceous binding molecule with immunoglobulin-like functions, as explained above. Thus, in an exemplary embodiment detecting the amount of e.g. S100A9 or S100A8 may be carried out using a first antibody or antibody fragment capable of specifically binding proSP-B, as well as a second antibody or antibody fragment capable of specifically binding the first antibody or antibody fragment. The documents cited above are incorporated herein by reference in their entirety. In case of conflict, the present specification, including definitions, will control.

The term “antibody” as used herein, is understood to include an immunoglobulin and an immunoglobulin fragment that is capable of specifically binding a selected protein, e.g. proSP-B, as well as a respective proteinaceous binding molecule with immunoglobulin-like functions. As an illustrative example an antibody may be a camel heavy chain immunoglobulin. As a few further non-limiting examples, an antibody may be an EGF-like domain, a Kringle-domain, a fibronectin type I domain, a fibronectin type II domain, a fibronectin type III domain, a PAN domain, a G1a domain, a SRCR domain, a Kunitz/Bovine pancreatic trypsin Inhibitor domain, tendamistat, a Kazal-type serine protease inhibitor domain, a Trefoil (P-type) domain, a von Willebrand factor type C domain, an Anaphylatoxin-like domain, a CUB domain, a thyroglobulin type I repeat, an LDL-receptor class A domain, a Sushi domain, a Link domain, a Thrombospondin type I domain, an immunoglobulin domain or a an immunoglobulin-like domain (see above for further examples). In some embodiments an antibody is an aptamer, including a Spiegelmer®, described in e.g. WO 01/92655. An aptamer is typically a nucleic acid molecule that can be selected from a random nucleic acid pool based on its ability to bind a selected other molecule such as a peptide, a protein, a nucleic acid molecule a or a cell. Aptamers, including Spiegelmers, are able to bind molecules such as peptides, proteins and low molecular weight compounds. Spiegelmers® are composed of L-isomers of natural oligonucleotides. Aptamers are engineered through repeated rounds of in vitro selection or through the SELEX (systematic evolution of ligands by exponential enrichment) technology. The affinity of Spiegelmers to their target molecules often lies in the pico- to nanomolar range and is thus comparable to immunoglobulins. An aptamer may also be a peptide. A peptide aptamer consists of a short variable peptide domain, attached at both ends to a protein scaffold. Throughout this document the term antibody may be used in conjunction with the term “proteinaceous binding partner”, even though the term “antibody” includes such a binding partner. This redundant twofold denomination is merely intended to take account of the frequent usage of the word “antibody” in the art, synonymously designating an immunoglobulin an antibody.

By “fragment” in reference to a polypeptide such as an immunoglobulin or a proteinaceous binding molecule is meant any amino acid sequence present in a corresponding polypeptide, as long as it is shorter than the full length sequence and as long as it is capable of performing the function of interest of the protein—in the case of an immunoglobulin specifically binding to the desired target, e.g. antigen (proSP-B, for example). The term “immunoglobulin fragment” refers to a portion of an immunoglobulin, often the hypervariable region and portions of the surrounding heavy and light chains that displays specific binding affinity for a particular molecule. A hypervariable region is a portion of an immunoglobulin that physically binds to the polypeptide target.

An immunoglobulin may be monoclonal or polyclonal. The term “polyclonal” refers to immunoglobulins that are heterogenous populations of immunoglobulin molecules derived from the sera of animals immunized with an antigen or an antigenic functional derivative thereof. For the production of polyclonal immunoglobulins, one or more of various host animals may be immunized by injection with the antigen. Various adjuvants may be used to increase the immunological response, depending on the host species. “Monoclonal immunoglobulins” or “Monoclonal antibodies” are substantially homogenous populations of immunoglobulins to a particular antigen. They may be obtained by any technique which provides for the production of immunoglobulin molecules by continuous cell lines in culture. Monoclonal immunoglobulins may be obtained by methods well known to those skilled in the art (see for example, Köhler et al., Nature (1975) 256, 495-497, and U.S. Pat. No. 4,376,110). An immunoglobulin or immunoglobulin fragment with specific binding affinity only for e.g. a region that corresponds to amino acid positions 63-79 of the human protein S100A9, for a region that corresponds to amino acid position 73-85 of the human protein S100A9 or a region that corresponds to amino acid positions 55-71 of the human protein S100A8 can be isolated, enriched, or purified from a prokaryotic or eukaryotic organism. Routine methods known to those skilled in the art enable production of both immunoglobulins or immunoglobulin fragments and proteinaceous binding molecules with immunoglobulin-like functions, in both prokaryotic and eukaryotic organisms.

In more detail, an immunoglobulin may be isolated by comparing its binding affinity to a protein of interest, e.g. S100A9 or S100A8, with its binding affinity to other polypeptides. Humanized forms of the antibodies may be generated using one of the procedures known in the art such as chimerization or CDR grafting. In general, techniques for preparing monoclonal antibodies and hybridomas are well known in the art. Any animal such as a goat, a mouse or a rabbit that is known to produce antibodies can be immunized with the selected polypeptide, e.g. a polypeptide with the sequence of a region that corresponds to amino acid positions 63-79 of the human protein S100A9, for a region that corresponds to amino acid position 73-85 of the human protein S100A9 or a region that corresponds to amino acid positions 55-71 of the human protein S100A8.

Methods for immunization are well known in the art. Such methods include subcutaneous or intraperitoneal injection of the polypeptide. One skilled in the art will recognize that the amount of polypeptide used for immunization and the immunization regimen will vary based on the animal which is immunized, including the species of mammal immunized, its immune status and the body weight of the mammal, as well as the antigenicity of the polypeptide and the site of injection.

The polypeptide may be modified or administered in an adjuvant in order to increase the peptide antigenicity. Methods of increasing the antigenicity of a polypeptide are well known in the art. Such procedures include coupling the antigen with a heterologous protein (such as globulin or β-galactosidase) or through the inclusion of an adjuvant during immunization.

Typically, the immunized mammals are bled and the serum from each blood sample is assayed for particular antibodies using appropriate screening assays. As an illustrative example, anti-S100A9 or anti-S100A8 immunoglobulins may be identified by immunoprecipitation of ¹²⁵I-labeled cell lysates from cells expressing a polypeptide with the sequence of a region that corresponds to amino acid positions 63-79 of the human protein S100A9, for a region that corresponds to amino acid position 73-85 of the human protein S100A9 or a region that corresponds to amino acid positions 55-71 of the human protein S100A8. Anti-S100A9 or anti-S100A8 immunoglobulins may also be identified by flow cytometry, e.g., by measuring fluorescent staining of Ramos cells incubated with an antibody believed to recognize anti-S100A9 or anti-S100A8.

For monoclonal immunoglobulins, lymphocytes, typically splenocytes, from the immunized animals are removed, fused with an immortal cell line, typically myeloma cells, such as SP2/0-Ag14 myeloma cells, and allowed to become monoclonal immunoglobulin producing hybridoma cells. Typically, the immortal cell line such as a myeloma cell line is derived from the same mammalian species as the lymphocytes. Illustrative immortal cell lines are mouse myeloma cell lines that are sensitive to culture medium containing hypoxanthine, aminopterin and thymidine (“HAT medium”). Typically, HAT-sensitive mouse myeloma cells are fused to mouse splenocytes using 1500 molecular weight polyethylene glycol (“PEG 1500”). Hybridoma cells resulting from the fusion may then be selected using HAT medium, which kills unfused and unproductively fused myeloma cells (unfused splenocytes die after several days because they are not transformed).

Any one of a number of methods well known in the art can be used to identify a hybridoma cell which produces an immunoglobulin with the desired characteristics. Typically the culture supernatants of the hybridoma cells are screened for immunoglobulins against the antigen. Suitable methods include, but are not limited to, screening the hybridomas with an ELISA assay, Western blot analysis, or radioimmunoassay (Lutz et al., Exp. Cell Res. [1988] 175, 109-124). Hybridomas prepared to produce anti-S100A9 or anti-S100A8 immunoglobulins may for instance be screened by testing the hybridoma culture supernatant for secreted antibodies having the ability to bind to a recombinant cell line expressing a polypeptide with the sequence of a region that corresponds to amino acid positions 63-79 of the human protein S100A9, for a region that corresponds to amino acid position 73-85 of the human protein S100A9 or a region that corresponds to amino acid positions 55-71 of the human protein S100A8. To produce antibody homologs which are within the scope of the invention, including for example, anti-S100A9 or anti-S100A8 antibody homologs, that are intact immunoglobulins, hybridoma cells that tested positive in such screening assays can be cultured in a nutrient medium under conditions and for a time sufficient to allow the hybridoma cells to secrete the monoclonal immunoglobulins into the culture medium. Tissue culture techniques and culture media suitable for hybridoma cells are well known in the art. The conditioned hybridoma culture supernatant may be collected and for instance the anti-S100A9 immunoglobulins or the anti-S100A8 immunoglobulins optionally further purified by well-known methods. Alternatively, the desired immunoglobulins may be produced by injecting the hybridoma cells into the peritoneal cavity of an unimmunized mouse. The hybridoma cells proliferate in the peritoneal cavity, secreting the immunoglobulin which accumulates as ascites fluid. The immunoglobulin may be harvested by withdrawing the ascites fluid from the peritoneal cavity with a syringe.

Hybridomas secreting the desired immunoglobulins are cloned and the class and subclass are determined using procedures known in the art. For polyclonal immunoglobulins, immunoglobulin containing antisera is isolated from the immunized animal and is screened for the presence of immunoglobulins with the desired specificity using one of the above-described procedures. The above-described antibodies may also be immobilized on a solid support. Examples of such solid supports include plastics such as polycarbonate, complex carbohydrates such as agarose and sepharose, acrylic resins and such as polyacrylamide and latex beads. Techniques for coupling antibodies to such solid supports are well known in the art.

A plurality of conventional display technologies is available to select an immunoglobulin, immunoglobulin fragment or proteinaceous binding molecule. Li et al. (Organic & Biomolecular Chemistry (2006), 4, 3420-3426) have for example demonstrated how a single-chain Fv fragment capable of forming a complex with a selected DNA adapter can be obtained using phage display. Display techniques for instance allow the generation of engineered immunoglobulins and ligands with high affinities for a selected target molecule. It is thus also possible to display an array of peptides or proteins that differ only slightly, typically by way of genetic engineering. Thereby it is possible to screen and subsequently evolve proteins or peptides in terms of properties of interaction and biophysical parameters. Iterative rounds of mutation and selection can be applied on an in vitro basis.

In vitro display technology for the selection of peptides and proteins relies on a physical linkage between the peptide or protein and a nucleic acid encoding the same. A large panel of techniques has been established for this purpose, with the most commonly used being phage/virus display, ribosome display, cell-surface display, ‘peptides on plasmids’, mRNA display, DNA display, and in vitro compartmentalisation including micro-bead display (for reviews see e.g. Rothe, A., et al., FASEB J. (2006) 20, 1599-1610; Sergeeva, A., et al., Advanced Drug Delivery Reviews (2006) 58, 1622-1654).

Different means of physically linking a protein or peptide and a nucleic acid are also available. Expression in a cell with a cell surface molecule, expression as a fusion polypeptide with a viral/phage coat protein, a stabilised in vitro complex of an RNA molecule, the ribosome and the respective polypeptide, covalent coupling in vitro via a puromycin molecule or via micro-beads are examples of ways of linking the protein/peptide and the nucleic acid presently used in the art. A further display technique relies on a water-in-oil emulsion. The water droplets serve as compartments in each of which a single gene is transcribed and translated (Tawfik, D. S., & Griffiths, A. D., Nature Biotech. (1998) 16, 652-656, US patent application 2007/0105117). This physical linkage between the peptide or protein and the nucleic acid (encoding it) provides the possibility of recovering the nucleic acid encoding the selected protein or peptide. Compared to techniques such as immunoprecipitation, in display techniques thus not only binding partners of a selected target molecule can be identified or selected, but the nucleic acid of this binding partner can be recovered and used for further processing. Present display techniques thus provide means for e.g. target discovery, lead discovery and lead optimisation. Vast libraries of peptides or proteins, e.g. antibodies, potentially can be screened on a large scale.

As indicated above, a detectable marker may be coupled to a binding partner of a polypeptide with the sequence of a region that corresponds to amino acid positions 63-79 of the human protein S100A9, for a region that corresponds to amino acid position 73-85 of the human protein S100A9 or a region that corresponds to amino acid positions 55-71 of the human protein S100A8, as the case may be, or a molecule that forms a complex with the binding partner of one of these peptides. A respective detectable marker, which may be coupled to a binding partner of one of these peptides, or a molecule that forms a complex therewith, may be an optically detectable label, a fluorophore, or a chromophore. Examples of suitable labels include, but are not limited to, an organic molecule, an enzyme, a radioactive, fluorescent, and/or chromogenic moiety, a luminescent moiety, a hapten, digoxigenin, biotin, a metal complex, a metal and colloidal gold. Accordingly an excitable fluorescent dye, a radioactive amino acid, a fluorescent protein or an enzyme may for instance be used to detect e.g. the level of S100A9 and/or S100A8, in which the region required for binding to the TLR4 receptor is accessible. Examples of suitable fluorescent dyes include, but are not limited to, fluorescein isothiocyanate, 5,6-carboxymethyl fluorescein, Cascade Blue®, Oregon Green®, Texas red, nitrobenz-2-oxa-1,3-diazol-4-yl, coumarin, dansyl chloride, rhodamine, amino-methyl coumarin, DAPI, Eosin, Erythrosin, BODIPY®, pyrene, lissamine, xanthene, acridine, an oxazine, phycoerythrin, a Cy dye such as Cy3, Cy3.5, Cy5, Cy5PE, Cy5.5, Cy7, Cy7PE or Cy7APC, an Alexa dye such as Alexa 647, and NBD (Naphthol basic dye). Examples of suitable fluorescent protein include, but are not limited to, EGFP, emerald, EYFP, a phycobiliprotein such as phycoerythrin (PE) or allophycocyanin, Monomeric Red Fluorescent Protein (mRFP), mOrange, mPlum and mCherry. In some embodiments a reversibly photoswitchable fluorescent protein such as Dronpa, bsDronpa and Padron may be employed (Andresen, M., et al., Nature Biotechnology (2008) 26, 9, 1035). Regarding suitable enzymes, alkaline phosphatase, soybean peroxidase, or horseradish peroxidase may serve as a few illustrative examples. In some embodiments a method of detection may include electrophoresis, HPLC, flow cytometry, fluorescence correlation spectroscopy or a modified form of these techniques. Some or all of these steps may be part of an automated separation/detection system.

An immunoglobulin or a proteinaceous binding partner as described in this document may in some embodiments be used in diagnosis of a condition associated with an inflammatory process in the organism of a subject. As explained above, accessibility of the region corresponding to amino acid positions 63-79 of the human protein S100A9, as well as the region corresponding to amino acid positions 73-85 of the human protein S100A9 and accessibility of the region corresponding amino acid positions 55-71 of the human protein S100A8 indicates that binding to the TLR4 receptor by S100A9 and S100A8 can occur, since the proteins are not in a heterotetrameric complex. Accordingly, an immunoglobulin or a proteinaceous binding partner with a binding specificity as defined above can be used to diagnose that a subject is suffering from an inflammatory condition, in which S100A9 and S100A8 are involved. Furthermore, typically at least some sites of inflammation in the organism of the subject can be identified.

In some embodiments a method of diagnosing an inflammatory condition by using an immunoglobulin or a proteinaceous binding partner with the above specificity involves the use of a molecular imaging technique. For this purpose the immunoglobulin or a proteinaceous binding partner may have a radioactive label. Two illustrative examples of a suitable radioactive label are ¹²⁴I and ⁸⁹Zr, which may be coupled to the immunoglobulin or a proteinaceous binding partner by means of a chelating moiety. In some embodiments ⁶⁸Ga may also be used as a radioactive label. Positron emission tomography (PET) imaging may then be used. A typical PET scanner that is used in the art can detect concentrations between 10⁻¹¹ M and 10⁻¹² M, which is sufficient for the detection of S100A9 and S100A8. PET can quantitatively image the distribution of a radiolabeled immunoglobulin or a proteinaceous binding partner within the organism of the subject. Further molecular imaging techniques that may be used include, but are not limited to, molecular magnetic resonance imaging (MRI), bioluminescence, fluorescence, targeted ultrasound, and single photon emission computed tomography (SPECT). An overview on molecular imaging techniques has been given by Dzik-Jurasz (The British Journal of Radiology (2003) 76 S98-S109). In some embodiments the immunoglobulin or proteinaceous binding partner may be coupled to a nanoparticle such as a nanocrystal.

Where desired, an immunoglobulin or a proteinaceous binding partner as defined above may be used in a hybrid imaging approach. For example, a PET/CT or a SPECT/CT camera is a commercially available combined system, which allows sequentially acquiring both anatomic and functional information that is accurately fused in a single examination. Integrated PET/magnetic resonance imaging allows a correction for motion of organs or subjects. Magnetic resonance imaging also offers information about perfusion and blood flow, which may be desired in PET reconstruction and data analysis in the context of inflammation. Molecular imaging by means of an immunoglobulin or a proteinaceous binding partner may also be carried out in the form of photoacoustic tomography (PAT) or combined with PAT. PAT is based on the conversion from optical to ultrasonic energy. Currently PAT is carried out by irradiating the biological tissue to be imaged using a nanosecond-pulsed laser beam to engender thermal and acoustic impulse responses. Today, PAT is generally implemented as focused-scanning photoacoustic microscopy (PAM), photoacoustic computed tomography (PACT), and photoacoustic endoscopy (PAE).

An immunoglobulin or a proteinaceous binding partner as disclosed in this document may in some embodiments be used in therapy, in particular in treating a condition, including a disease, associated with an inflammatory process in the organism of a subject. An immunoglobulin or a proteinaceous binding partner as disclosed in this document may also be used in preventing a condition associated with an inflammatory process in the organism of a subject. The term “preventing” refers to decreasing the probability that an organism contracts or develops an abnormal condition. In some embodiments such an immunoglobulin or proteinaceous binding partner is used in preventing or treating chronic or acute aseptic inflammation, neuropathic pain, primary graft failure, ischemia-reperfusion injury, reperfusion injury, reperfusion edema, allograft dysfunction, pulmonary reimplantation response and/or primary graft dysfunction in organ transplantation in a subject in need thereof. An immunoglobulin or a proteinaceous binding partner as disclosed in this document may also be used in the treatment of septic shock, asthmatic conditions, Crohn's disease, ulcerous colitis, reperfusion injury, auto-immune diseases, inflammatory bowel disease, atherosclerosis, restenosis, coronary heart disease, diabetes, rheumatoidal diseases, dermatological diseases, such as psoriasis and seborrhea, graft rejection, and inflammation of the lungs, heart, kidney, oral cavity (e.g., periodontitis) or uterus. It is understood that the immunoglobulin or a proteinaceous binding partner may also find use in diagnosis of such a condition.

A respective method includes administering an immunoglobulin or a proteinaceous binding partner as disclosed herein. In some embodiments the immunoglobulin or proteinaceous binding partner may be administered in combination with a TLR4 inhibitor. In some embodiments the immunoglobulin or proteinaceous binding partner may be administered in combination with a TLR2, a MYD88, a TICAMI and/or a TIRAP inhibitor.

“Treating” or “treatment” or “alleviation” refers to both therapeutic treatment and prophylactic or preventative measures, wherein the object is to prevent, slow down (lessen) or at least partially alleviate or abrogate an abnormal, including pathologic, condition in the organism. Those in need of treatment include those already with the disorder as well as those prone to having the disorder or those in whom the disorder is to be prevented (prophylaxis). The term “administering” relates to a method of incorporating a compound into cells or tissues of an organism.

As explained above, in some embodiments there is provided a peptide or a combination of peptides. Where a peptide is provided, the peptide is isolated. Likewise where a combination of peptides is provided, the peptides of the combination of peptides are isolated. The term “isolated” indicates that the peptide(s) or nucleic acid molecule(s) has/have been removed from its/their normal physiological environment, e.g. a natural source, or that a peptide or nucleic acid is synthesized. Use of the term “isolated” indicates that a naturally occurring sequence has been removed from its normal cellular, e.g. chromosomal, environment. Thus, the sequence may be in a cell-free medium or placed in a different cellular environment. Thus, a cell or cells may be included in a different medium such as an aqueous solution than provided originally, or placed in a different physiological environment. Typically isolated cells, peptides or nucleic acid molecule(s) constitute a higher fraction of the total cells, peptides or nucleic acid molecule(s) present in their environment, e.g. solution/suspension as applicable, than in the environment from which they were taken. By “isolated” in reference to a polypeptide or nucleic acid molecule is meant a polymer of amino acids (2 or more amino acids) or nucleotides coupled to each other, including a polypeptide or nucleic acid molecule that is isolated from a natural source or that is synthesized. The term “isolated” does not imply that the sequence is the only amino acid chain or nucleotide chain present, but that it is essentially free, e.g. about 90-95% pure or more, of e.g. non-amino acid material and/or non-nucleic acid material, respectively, naturally associated with it.

As indicated above, instead of or in addition to peptides, peptidomimetics may likewise be used in the context of the present invention. The term “peptidomimetic” as used herein refers to a compound that has the same general structure as a corresponding polypeptide, but which includes modifications that increase its stability or biological function. In some embodiments a peptidomimetic may include one or more D-amino acids, essentially consist of D-amino acids or consist of D-amino acids. D-amino acids are the optical isomer of a naturally occurring L amino acid. A D amino acid can be taken to be a mirror image of a L amino acid. Stretches of D amino acids are less prone to be degraded in a host organism via proteolysis. In some embodiments a peptidomimetic may be an inverso analog, which is an analog of the same sequence that consists only of D amino acids. In some embodiments a peptidomimetic may be a “reverso” analogue of a given peptide, which means that the peptidomimetic includes the reverse sequence of the peptide. In some embodiments a peptidomimetic may be a “D-retro-enantiomer peptide”, which is an analog that consists of D-amino acids, with the sequence arranged in the reversed order. A peptidomimetic may also include, essentially consist of or consist of a peptoid. A peptoid differs from peptides in that the side chain is connected to the amide nitrogen rather than the a carbon atom. A peptoid can thus be taken to be an oligo(N-alkyl)glycine, which nevertheless has the same or substantially the same amino acid sequence as the corresponding polypeptide. Peptoids are typically resistant to proteases and other modifying enzymes and can have a much higher cell permeability than peptides, see e.g. Kwon, Y.-U., and Kodadek, T., J. Am. Chem. Soc. (2007) 129, 1508-1509. This document is incorporated herein by reference in its entirety. In case of conflict, the present specification, including definitions, will control.

The peptide or peptidomimetic may be prepared by any method, such as by synthesizing the peptide or peptidomimetic, or by expressing a nucleic acid encoding an appropriate amino acid sequence in a cell and harvesting the peptide from the cell. A combination of such methods may likewise be used. Methods of de novo synthesizing peptides and peptidomimetics, and methods of recombinantly producing peptides and peptidomimetics are well known in the art.

The peptide or peptidomimetic, or the combination of peptides or peptidomimetics as disclosed herein may capable of interfering with the binding of a S100A8 protein and/or a S100A9 protein to a TLR4 receptor. Where the TLR4 receptor is present on the surface of a cell, as a result, cellular signalling induced by the binding of the respective S100A8 protein to the TLR4 receptor may likewise be induced. The terms “signalling” and “signal transduction pathway” refer to cellular mechanisms and to molecules that act on cellular components in response to a certain condition, change or external stimulus. Typically such mechanisms and molecules propagate an extracellular signal through the cell membrane to become an intracellular signal. This signal can then stimulate a cellular response.

A nucleic acid molecule as disclosed herein may contain one or more sequences that encode one or more peptides/proteins. In some embodiments among these encoded sequences, or this encoded sequence, is a sequence that encodes the sequence of SEQ ID NO: 6 or a homolog thereof. In some embodiments among these encoded sequences, or this encoded sequence, is a sequence that encodes the sequence of SEQ ID NO: 9 or a homolog thereof. In some embodiments among these encoded sequences, or this encoded sequence, is a sequence that encodes the sequence of SEQ ID NO: 12 or a homolog thereof. In some embodiments among these encoded sequences, or this encoded sequence, is a sequence that encodes both the sequence of SEQ ID NO: 6 or a homolog thereof and a sequence that encodes both the sequence of SEQ ID NO: 12 or a homolog thereof. In some embodiments among these encoded sequences, or this encoded sequence, is a sequence that encodes both the sequence of SEQ ID NO: 9 or a homolog thereof and a sequence that encodes both the sequence of SEQ ID NO: 12 or a homolog thereof. In some embodiments among these encoded sequences, or this encoded sequence, is a sequence that encodes both the sequence of SEQ ID NO: 6 or a homolog thereof and a sequence that encodes both the sequence of SEQ ID NO: 9 or a homolog thereof.

In some embodiments a nucleic acid molecule as disclosed herein contains a single sequence encoding a peptide that contains the sequence of SEQ ID NO: 6 or a homolog thereof. In some embodiments a nucleic acid molecule as disclosed herein contains a single sequence that encodes a peptide, which has a length of 60 amino acids or less that contains the sequence of SEQ ID NO: 6 or a homolog thereof. In some embodiments a nucleic acid molecule as disclosed herein contains a single sequence that encodes a peptide, which has a length of 50 amino acids or less that contains the sequence of SEQ ID NO: 6 or a homolog thereof. In some embodiments a nucleic acid molecule as disclosed herein contains a single sequence that encodes a peptide, which has a length from 18-50 amino acids that contains the sequence of SEQ ID NO: 6 or a homolog thereof. In some embodiments a nucleic acid molecule as disclosed herein contains a single sequence that encodes a peptide, which has a length from 20-50 amino acids that contains the sequence of SEQ ID NO: 6 or a homolog thereof. In some embodiments a nucleic acid molecule as disclosed herein contains a single sequence that encodes a peptide, which has a length of 40 amino acids or less that contains the sequence of SEQ ID NO: 6 or a homolog thereof. In some embodiments a nucleic acid molecule contains a single sequence that encodes a peptide, which has a length from 20-40 amino acids that contains the sequence of SEQ ID NO: 6 or a homolog thereof. In some embodiments a nucleic acid molecule as disclosed herein contains a single sequence that encodes a peptide, which has a length of 30 amino acids or less that contains the sequence of SEQ ID NO: 6 or a homolog thereof. In some embodiments a nucleic acid molecule as disclosed herein contains a single sequence that encodes a peptide, which has a length from 18-30 amino acids that contains the sequence of SEQ ID NO: 6 or a homolog thereof. In some embodiments a nucleic acid molecule as disclosed herein contains a single sequence that encodes a peptide, which has a length from 20-30 amino acids that contains the sequence of SEQ ID NO: 6 or a homolog thereof. In some embodiments a nucleic acid molecule as disclosed herein contains a single sequence that encodes a peptide that essentially consists of the sequence of SEQ ID NO: 6 or a homolog thereof. In some embodiments a nucleic acid molecule contains a single sequence that encodes a peptide that consists of the sequence of SEQ ID NO: 6 or a homolog thereof.

In some embodiments a nucleic acid molecule contains a single sequence encoding a peptide that contains the sequence of SEQ ID NO: 9 or a homolog thereof. In some embodiments a nucleic acid molecule as disclosed herein contains a single sequence that encodes a peptide, which has a length of 60 amino acids or less that contains the sequence of SEQ ID NO: 9 or a homolog thereof. In some embodiments a nucleic acid molecule contains a single sequence that encodes a peptide, which has a length of 50 amino acids or less that contains the sequence of SEQ ID NO: 9 or a homolog thereof. In some embodiments a nucleic acid molecule contains a single sequence that encodes a peptide, which has a length from 14-50 amino acids that contains the sequence of SEQ ID NO: 9 or a homolog thereof. In some embodiments a nucleic acid molecule as disclosed herein contains a single sequence that encodes a peptide, which has a length from 20-50 amino acids that contains the sequence of SEQ ID NO: 9 or a homolog thereof. In some embodiments a nucleic acid molecule contains a single sequence that encodes a peptide, which has a length of 40 amino acids or less that contains the sequence of SEQ ID NO: 9 or a homolog thereof. In some embodiments a nucleic acid molecule contains a single sequence that encodes a peptide, which has a length from 14-40 amino acids that contains the sequence of SEQ ID NO: 9 or a homolog thereof. In some embodiments a nucleic acid molecule contains a single sequence that encodes a peptide, which has a length from 20-40 amino acids that contains the sequence of SEQ ID NO: 9 or a homolog thereof. In some embodiments a nucleic acid molecule as disclosed herein contains a single sequence that encodes a peptide, which has a length of 30 amino acids or less that contains the sequence of SEQ ID NO: 9 or a homolog thereof. In some embodiments a nucleic acid molecule contains a single sequence that encodes a peptide, which has a length from 14-30 amino acids that contains the sequence of SEQ ID NO: 9 or a homolog thereof. In some embodiments a nucleic acid molecule contains a single sequence that encodes a peptide, which has a length from 20-30 amino acids that contains the sequence of SEQ ID NO: 9 or a homolog thereof. In some embodiments a nucleic acid molecule contains a single sequence that encodes a peptide, which has a length of 28 amino acids or less, such as 25 amino acids or less, 24 amino acids or less, 23 amino acids or less, 22 amino acids or less or 21 amino acids or less. In some embodiments a nucleic acid molecule as disclosed herein contains a single sequence that encodes a peptide, which has a length of 20 amino acids or less that contains the sequence of SEQ ID NO: 9 or a homolog thereof. In some embodiments a nucleic acid molecule contains a single sequence that encodes a peptide, which has a length from 14-20 amino acids that contains the sequence of SEQ ID NO: 9 or a homolog thereof. In some embodiments a nucleic acid molecule contains a single sequence that encodes a peptide that essentially consists of the sequence of SEQ ID NO: 9 or a homolog thereof. In some embodiments a nucleic acid molecule as disclosed herein contains a single sequence that encodes a peptide that consists of the sequence of SEQ ID NO: 9 or a homolog thereof.

In some embodiments a nucleic acid molecule as disclosed herein contains a single sequence encoding a peptide that contains the sequence of SEQ ID NO: 12 or a homolog thereof. In some embodiments a nucleic acid molecule contains a single sequence that encodes a peptide, which has a length of 60 amino acids or less that contains the sequence of SEQ ID NO: 12 or a homolog thereof. In some embodiments a nucleic acid molecule contains a single sequence that encodes a peptide, which has a length of 50 amino acids or less that contains the sequence of SEQ ID NO: 12 or a homolog thereof. In some embodiments a nucleic acid molecule contains a single sequence that encodes a peptide, which has a length from 18-50 amino acids that contains the sequence of SEQ ID NO: 12 or a homolog thereof. In some embodiments a nucleic acid molecule as disclosed herein contains a single sequence that encodes a peptide, which has a length from 20-50 amino acids that contains the sequence of SEQ ID NO: 12 or a homolog thereof. In some embodiments a nucleic acid molecule contains a single sequence that encodes a peptide, which has a length of 40 amino acids or less that contains the sequence of SEQ ID NO: 12 or a homolog thereof. In some embodiments a nucleic acid molecule as disclosed herein contains a single sequence that encodes a peptide, which has a length from 18-40 amino acids that contains the sequence of SEQ ID NO: 12 or a homolog thereof. In some embodiments a nucleic acid molecule contains a single sequence that encodes a peptide, which has a length from 20-40 amino acids that contains the sequence of SEQ ID NO: 12 or a homolog thereof. In some embodiments a nucleic acid molecule contains a single sequence that encodes a peptide, which has a length of 30 amino acids or less that contains the sequence of SEQ ID NO: 12 or a homolog thereof. In some embodiments a nucleic acid molecule contains a single sequence that encodes a peptide, which has a length from 18-30 amino acids that contains the sequence of SEQ ID NO: 12 or a homolog thereof. In some embodiments a nucleic acid molecule as disclosed herein contains a single sequence that encodes a peptide, which has a length from 20-30 amino acids that contains the sequence of SEQ ID NO: 12 or a homolog thereof. In some embodiments a nucleic acid molecule contains a single sequence that encodes a peptide that essentially consists of the sequence of SEQ ID NO: 12 or a homolog thereof. In some embodiments a nucleic acid molecule contains a single sequence that encodes a peptide that consists of the sequence of SEQ ID NO: 12 or a homolog thereof.

The term “nucleic acid” as used herein refers to any nucleic acid molecule in any possible configuration, such as single stranded, double stranded or a combination thereof.

Nucleic acids include for instance DNA molecules, RNA molecules, analogues of the DNA or RNA generated using nucleotide analogues or using nucleic acid chemistry, locked nucleic acid molecules (LNA), protein nucleic acids molecules (PNA) and tecto-RNA molecules (e.g. Liu, B., et al., J. Am. Chem. Soc. (2004) 126, 4076-4077). A PNA molecule is a nucleic acid molecule in which the backbone is a pseudopeptide rather than a sugar. Accordingly, PNA generally has a charge neutral backbone, in contrast to for example DNA or RNA. Nevertheless, PNA is capable of hybridising at least complementary and substantially complementary nucleic acid strands, just as e.g. DNA or RNA (to which PNA is considered a structural mimic). An LNA molecule has a modified RNA backbone with a methylene bridge between C4′ and O2′, which locks the furanose ring in a N-type configuration, providing the respective molecule with a higher duplex stability and nuclease resistance. Unlike a PNA molecule an LNA molecule has a charged backbone. DNA or RNA may be of genomic or synthetic origin and may be single or double stranded. Such nucleic acid can be e.g. mRNA, cRNA, synthetic RNA, genomic DNA, cDNA, synthetic DNA, a copolymer of DNA and RNA, oligonucleotides, etc. A respective nucleic acid may furthermore contain non-natural nucleotide analogues and/or be linked to an affinity tag or a label.

Many nucleotide analogues are known and can be used in a method disclosed herein. A nucleotide analogue is a nucleotide containing a modification at for instance the base, sugar, or phosphate moieties. As an illustrative example, a substitution of 2′-OH residues of siRNA with 2′F, 2′O-Me or 2′H residues is known to improve the in vivo stability of the respective RNA. Modifications at the base moiety include natural and synthetic modifications of A, C, G, and T/U, different purine or pyrimidine bases, such as uracil-5-yl, hypoxanthin-9-yl, and 2-aminoadenin-9-yl, as well as non-purine or non-pyrimidine nucleotide bases. Other nucleotide analogues serve as universal bases. Universal bases include 3-nitropyrrole and 5-nitroindole. Universal bases are able to form a base pair with any other base. Base modifications often can be combined with for example a sugar modification, such as for instance 2′-O-methoxyethyl, e.g. to achieve unique properties such as increased duplex stability.

In some embodiments a nucleic acid molecule as disclosed herein is capable of expressing the sequence of SEQ ID NO: 6 or a homolog thereof, the sequence of SEQ ID NO: 9 or a homolog thereof and/or the sequence of SEQ ID NO: 12 or a homolog thereof. In some embodiments a nucleic acid molecule includes a sequence that allows the sequence of SEQ ID NO: 6 or a homolog thereof, the sequence of SEQ ID NO: 9 or a homolog thereof and/or the sequence of SEQ ID NO: 12 or a homolog thereof to be expressed. The nucleic acid molecule may for instance include a promoter operatively linked to one or more of these sequences, or to a sequence that includes one or more of these sequences. In some embodiments a nucleic acid molecule as disclosed herein includes a termination signal operatively linked to one or more of these sequences, or to a sequence that includes one or more of these sequences. In some embodiments a nucleic acid molecule according to the invention includes a regulatory sequence operatively linked to one or more of these sequences, or to a sequence that includes one or more of these sequences.

The term “regulatory sequence” includes controllable transcriptional promoters, operators, enhancers, silencers, transcriptional terminators, 5′ and 3′ untranslated regions which interact with host cellular proteins to carry out transcription and translation and other elements that may control gene expression including initiation and termination codons. The regulatory sequences can be native (homologous), or can be foreign (heterologous) to the cell and/or the nucleotide sequence that is used. The precise nature of the regulatory sequences needed for gene sequence expression may vary from organism to organism, but shall in general include a promoter region which, in prokaryotes, contains both the promoter (which directs the initiation of RNA transcription) as well as the DNA sequences which, when transcribed into RNA, will signal synthesis initiation. Such regions will normally include those 5′-non-coding sequences involved with initiation of transcription and translation, such as the TATA box, capping sequence or CAAT sequence. These regulatory sequences are generally individually selected for a certain embodiment, for example for a certain cell to be used. The skilled artisan will be aware that proper expression in a prokaryotic cell also requires the presence of a ribosome-binding site upstream of the gene sequence-encoding sequence.

In some embodiments a nucleic acid molecule as disclosed herein is being expressed in a cell in order to obtain a peptide with the sequence of SEQ ID NO: 6 or a homolog thereof, the sequence of SEQ ID NO: 9 or a homolog thereof and/or the sequence of SEQ ID NO: 12 or a homolog thereof. In some embodiments the cell expresses a S100A9 protein, and/or a S100A8 protein. As explained below, expression of such a peptide may include the generation of a vector that has a construct with a sequence encoding the peptide. Once the vector or nucleic acid molecule that contains the construct(s) has been prepared for expression, the nucleic acid constructs) may be introduced into a selected suitable host cell by any of a variety of suitable means, i.e., transformation, transfection, conjugation, protoplast fusion, electroporation, particle gun technology, calcium phosphate-precipitation, direct microinjection, and the like. After the introduction of the vector, recipient cells are grown in a selective medium, which selects for the growth of vector-containing cells. Expression of the cloned gene(s) results in the production of a protein or peptide as disclosed herein, or fragments thereof. This can take place in the transformed cells as such, or following the induction of these cells to differentiate. A variety of incubation conditions can be used to form a peptide as disclosed herein. It may be desired to use conditions that mimic physiological conditions.

The terms “expression” and “expressed”, as used herein, are used in their broadest meaning, to signify that a sequence included in a nucleic acid molecule and encoding a peptide/protein is converted into its peptide/protein product. Thus, where the nucleic acid is DNA, expression refers to the transcription of a sequence of the DNA into RNA and the translation of the RNA into protein. Where the nucleic acid is RNA, expression may include the replication of this RNA into further RNA copies and/or the reverse transcription of the RNA into DNA and optionally the transcription of this DNA into further RNA molecule(s). In any case expression of RNA includes the translation of any of the RNA species provided/produced into protein. Hence, expression is performed by translation and includes one or more processes selected from the group consisting of transcription, reverse transcription and replication. Expression of the protein or peptide of the member of the plurality of peptides and/or proteins may be carried out using an in vitro expression system. Such an expression system may include a cell extract, typically from bacteria, rabbit reticulocytes or wheat germ. Many suitable systems are commercially available. The mixture of amino acids used may include synthetic amino acids if desired, to increase the possible number or variety of proteins produced in the library. This can be accomplished by charging tRNAs with artificial amino acids and using these tRNAs for the in vitro translation of the proteins to be selected. A nucleic acid molecule, such as DNA, is said to be “capable of expressing” a peptide/protein if it contains nucleotide sequences which contain transcriptional and translational regulatory information and such sequences are operably linked to nucleotide sequences which encode the polypeptide. A suitable embodiment for expression purposes is the use of a vector, in particular an expression vector. Thus, provided is also a host cell transformed/transfected with an expression vector.

In some embodiments a nucleic acid molecule as disclosed herein includes an expression cassette capable of inducing and/or regulating the expression of a peptide with the sequence of SEQ ID NO: 6 or a homolog thereof, the sequence of SEQ ID NO: 9 or a homolog thereof and/or the sequence of SEQ ID NO: 12 or a homolog thereof. In some embodiments a nucleic acid molecule as disclosed herein is encompassed by a vector that contains a promoter effective to initiate transcription in the respective host cell (whether of endogenous or exogenous origin).

As used herein, the term “expression cassette” refers to a nucleic acid molecule capable of directing expression of a particular nucleotide sequence in an appropriate host cell. An expression cassette includes a promoter operatively linked to the nucleotide sequence of interest, which is operatively linked to one or more termination signals. It may also include sequences required for proper translation of the nucleotide sequence. The coding region can encode a polypeptide of interest and can also encode a functional RNA of interest, including but not limited to, antisense RNA or a non-translated RNA, in the sense or antisense direction. The expression cassette comprising the nucleotide sequence of interest can be chimeric, meaning that at least one of its components is heterologous with respect to at least one of its other components. The expression cassette can also be one that is naturally occurring but has been obtained in a recombinant form useful for heterologous expression. In some embodiments, however, the expression cassette is heterologous with respect to the host; i.e., the particular nucleic acid sequence of the expression cassette does not occur naturally in the host cell and was introduced into the host cell or an ancestor of the host cell by a transformation event. The expression of the nucleotide sequence in the expression cassette can be under the control of a constitutive promoter or of an inducible promoter that initiates transcription only when the host cell is exposed to some particular external stimulus. In the case of a multicellular organism such as a plant or an animal, the promoter can also be specific to a particular tissue, organ, or stage of development.

By “gene” is meant a unit of inheritance that occupies a specific locus on a chromosome and that is a segment of nucleic acid associated with a biological function. A gene encompasses transcriptional and/or translational regulatory sequences as well as a coding region. Besides a coding sequence a gene may include a promoter region, a cis-regulatory sequence, a non-expressed DNA segment that is a specific recognition sequence for regulatory proteins, a non-expressed DNA segment that contributes to gene expression, a DNA segment designed to have desired parameters, or combinations thereof. A gene can be obtained by a variety of methods, including cloning from a biological sample, synthesis based on known or predicted sequence information, and recombinant derivation of an existing sequence.

The term “vector”, sometimes also referred to as gene delivery system or gene transfer vehicle, relates to a macromolecule or complex of molecules that include(s) a polynucleotide to be delivered to a host cell, whether in vitro, ex vivo or in vivo. Typically a vector is a single or double-stranded circular nucleic acid molecule that allows or facilitates the transfer of a nucleic acid sequence into a cell. A vector can generally be transfected into cells and replicated within or independently of a cell genome. A circular double-stranded nucleic acid molecule can be cut and thereby linearized upon treatment with restriction enzymes. An assortment of nucleic acid vectors, restriction enzymes, and the knowledge of the nucleotide sequences cut by restriction enzymes are readily available to those skilled in the art. A nucleic acid molecule encoding a peptide, such as a sequence that includes a sequence of SEQ ID NO: 6 or a homolog thereof, of SEQ ID NO: 9 or a homolog thereof and/or a sequence of SEQ ID NO: 12, or a homolog thereof, can be inserted into a vector by cutting the vector with restriction enzymes and ligating the two pieces together. A vector may for instance be a viral vector, such as a retroviral vector, a Lentiviral vector, a herpes virus based vector or an adenoviral vector. A vector may also be a plasmid vector, which is also a typical example of a prokaryotic vector. A respective plasmid may in some embodiments be a plasmid capable of replication in E. coli, such as, for example, pBR322, ColE1, pSC101, pACYC 184 or mVX. Bacillus plasmids include pC194, pC221 or pT127. Suitable Streptomyces plasmids include p1J101, and streptomyces bacteriophages such as φC31. A vector may also be a liposome-based extrachromosomal vector, also called episomal vector. Two illustrative examples of an episomal vector are an oriP-based vector and a vector encoding a derivative of EBNA-1. Lymphotrophic herpes virus is a herpes virus which replicates in a lymphoblast and becomes a plasmid for a part of its natural life-cycle. A vector may also be based on an organically modified silicate. In some embodiments a vector may be a transposon-based system, i.e. a transposon/transposase system, such as the so called Sleeping Beauty, the Frog Prince transposon—transposase system or the TTAA-specific transposon piggyBac system. Transposons are mobile genetic elements in that they are sequences of DNA that can move around to different positions within the genome of a single cell, a process called transposition. In the process, a transposon can cause mutations and change the amount of DNA in the genome.

The term “promoter” as used throughout this document, refers to a nucleic acid sequence needed for gene sequence expression. Promoter regions vary from organism to organism, but are well known to those skilled in the art for different organisms. For example, in prokaryotes, the promoter region contains both the promoter (which directs the initiation of RNA transcription) as well as the DNA sequences which, when transcribed into RNA, will signal synthesis initiation. Such regions will normally include those 5′-non-coding sequences involved with initiation of transcription and translation, such as the TATA box, capping sequence, CAAT sequence, and the like. Both constitutive and inducible promoters can be used in the context of the present invention, in accordance with the needs of a particular embodiment. A large number of promoters recognized by a variety of potential host cells are well known. The selected promoter can be operably linked to cistron DNA encoding a polypeptide described herein by removing the promoter from the source DNA via restriction enzyme digestion and inserting the isolated promoter sequence into the vector of choice. Both the native promoter sequence and many heterologous promoters may be used to direct amplification and/or expression of a selected nucleic acid sequence.

In a method disclosed herein a nucleic acid may be introduced into a host cells by any suitable technique of nucleic acid delivery for transformation of a cell available in the art. Examples of suitable techniques include, but are not limited to, direct delivery of DNA, e.g. via transfection, injection, including microinjection, electroporation, calcium phosphate precipitation, by using DEAE-dextran followed by polyethylene glycol, direct sonic loading, liposome mediated transfection, receptor-mediated transfection, microprojectile bombardment, agitation with silicon carbide fibers, Agrobacterium-mediated transformation, desiccation/inhibition-mediated DNA uptake or any combination thereof.

A method as disclosed herein may further include measuring the expression of a sequence that includes a sequence of SEQ ID NO: 6 or a homolog thereof, a sequence of SEQ ID NO: 9 or a homolog thereof and/or a sequence of SEQ ID NO: 12 or a homolog thereof. This can for instance be achieved by determining the number of RNA molecules transcribed from an encoding nucleic acid molecule that is under the control of a selected promoter. A method commonly used in the art is the subsequent copy of RNA to cDNA using reverse transcriptase and the coupling of the cDNA molecules to a fluorescent dye. The analysis may for example be performed in form of a DNA microarray. Numerous respective services and kits are commercially available, for instance GeneChip® expression arrays from Affymetrix. Other means of determining gene expression of a transcription factor include, but are not limited to, oligonucleotide arrays, and quantitative Real-time Polymerase Chain Reaction (RT-PCR).

In some embodiments it may be advantageous or desired to calibrate peptide/protein expression data or to rate them. Thus, in some embodiments a method as disclosed herein additionally includes the comparison of obtained results with those of one or more control measurements. Such a control measurement may include any condition that varies from the main measurement itself. It may include conditions of the method under which for example no expression of the respective peptide/protein occurs. A further means of a control measurement is the use of a mutated form of a respective peptide/protein, for example a nucleic acid sequence or gene not encoding the corresponding peptide/protein that includes the sequence of sequence of SEQ ID NO: 6 or a homolog thereof, the sequence of SEQ ID NO: 9 or a homolog thereof and/or the sequence of SEQ ID NO: 12 or a homolog thereof, or encoding a non-functional peptide/protein.

On a general basis the present invention also relates to methods and uses of diagnosing and methods and uses of treating a S100A8 and/or S100A9 mediated disorder, i.e. a disorder, condition, or disease state characterized by TLR4 signalling, including excessive TLR4 signalling, induced by one or both of the proteins S100A8 and S100A9. In a specific aspect, the TLR4 signalling is a level of TLR4 signalling in a cell or tissue suspected of being diseased that exceeds the level of TLR4 signalling in a similar non-diseased cell or tissue. In a specific aspect, a S100A8 and/or S100A9 mediated disorder includes an inflammation. In some embodiments the use of a peptide or peptidomimetic as disclosed herein allows blocking or reducing the TLR4 signalling activity.

In some methods and uses as disclosed herein the formation of a complex between S100A8 and/or S100A9 and a TLR4 receptor is reduced, including prevented. In some methods and uses as disclosed herein the formation of a heterotetrameric complex between S100A8 and S100A9 is reduced, including prevented.

In some embodiments a method disclosed herein includes a measurement of the formation of a complex between S100A8 and/or S100A9, or a functional fragment of one of these proteins, and a TLR4 receptor, or a functional fragment of a TLR4 receptor. In the context of binding to a TLR4 receptor, a functional fragment of S100A8 and a functional fragment of S100A9 are defined by two criteria. Firstly, a functional fragment is able to bind to and form a complex with a TLR4 receptor that is stable enough to affect signal transduction of the TLR4 receptor. Generally such a fragment of S100A8 contains an epitope with an amino acid sequence of a region that corresponds to the amino acid sequence ranging from amino acid position 55 to amino acid position 71 of the human S100A8 protein. Such a fragment of S100A9 generally contains an epitope with an amino acid sequence of a region that corresponds to the amino acid sequence ranging from amino acid position 63 to amino acid position 79 and/or ranging from amino acid position 73 to amino acid position 85 of the human S100A8 protein. Secondly, such a fragment may have at least 60% sequence identity with the corresponding amino acid sequence of a naturally existing variant of S100A8 and of S100A9, respectively. In some embodiments, a respective fragment has at least 80%, such at least 95% sequence identity with the corresponding amino acid sequence of a known variant of S100A8 and of S100A9, respectively. It is understood that a functional fragment of S100A8 or of S100A9 is able to be modulated by a compound in such a way that its complex formation with a TLR4 receptor is affected.

A functional fragment of the TLR4 receptor is defined by two criteria. Firstly, a functional fragment is able to bind to and form a complex with a S100A8 protein and a S100A9 protein that is stable enough to affect signal transduction of the TLR4 receptor. Secondly, such a fragment may have at least 60% sequence identity with the corresponding amino acid sequence of a naturally existing variant of the TLR4 receptor. In some embodiments, a respective fragment has at least 80%, such at least 95% sequence identity with the corresponding amino acid sequence of a known variant of the TLR4 receptor. It is understood that a functional fragment of the TLR4 receptor is able to be modulated by a compound in such a way that its complex formation with a S100A8 protein and a S100A9 protein is affected.

In some embodiments a method as disclosed herein includes a measurement of the bimolecular binding, i.e. the formation of a complex between a S100A8 protein or a functional fragment of a S100A8 protein, and a S100A9 protein, or a functional fragment of S100A9. In some embodiments a method includes a measurement of the tetramolecular binding, i.e. the formation of a complex between two molecules of S100A8 or a functional fragment of S100A8, and two molecules of S100A9, or a functional fragment of S100A9.

In the context of binding to each other, a functional fragment of S100A8 and a functional fragment of S100A9 are defined by three criteria. Firstly, a functional fragment of a S100A9 protein is able to bind to and form a complex with a S100A8 protein that is stable enough to be detected over more than a millisecond. Likewise, a functional fragment of a S100A8 protein is able to bind to and form a complex with a S100A9 protein that is stable enough to be detected over more than a millisecond. Generally a respective complex has a half-life of more than a millisecond under physiological conditions. Secondly, such a fragment is capable of binding a calcium ion. A respective fragment may also be able to bind a zinc and/or a copper ion. Typically, such a fragment of a S100A8 protein and of a S100A9 protein has at least one functional EF hand, i.e. an EF hand that contains the conserved amino acids known to be required for calcium binding. Thirdly, such a fragment may have at least 60% sequence identity with the corresponding amino acid sequence of a naturally existing variant of S100A8 and of S100A9, respectively. In some embodiments, a respective fragment has at least 80%, such at least 95% sequence identity with the corresponding amino acid sequence of a known variant of S100A8 and of S100A9, respectively. It is understood that a functional fragment of S100A8 and of S100A9, respectively, is able to be modulated by a compound in such a way that its complex formation with S100A9 and of S100A8, respectively, is affected.

Such a measurement of a complex formation may for instance rely on spectroscopical, photochemical, photometric, fluorometric, radiological, enzymatic or thermodynamic means, or on cellular effects. An example of a spectroscopical detection method is fluorescence correlation spectroscopy. A photochemical method is for instance photochemical cross-linking. The use of photoactive, fluorescent, radioactive or enzymatic labels, respectively, are examples for photometric, fluorometric, radiological and enzymatic detection methods. An example of a thermodynamic detection method is isothermal titration calorimetry. An example of a method using cellular effects is the measurement of the release of an inflammatory factor from a monocyte, for example the release of TNFα. Some of these methods may include additional separation techniques such as electrophoresis or HPLC. In detail, examples for the use of a label may include a compound as a probe or an immunoglobulin with an attached enzyme, the reaction catalysed by which leads to a detectable signal. An example of a method using a radioactive label and a separation by electrophoresis is an electrophoretic mobility shift assay.

A measurement of a complex formation between a S100A9 and a S100A8 protein or a respective fragment, or between a S100A9 and/or a S100A8 protein or a respective fragment may be included in a method of identifying a compound suitable for diagnosis, prevention and/or treatment of a condition associated with an inflammatory state in an organism. The formation of a complex may be analysed on the basis of the molecular weight of the target of an immunoglobulin, or a binding partner with immunoglobulin-like functions, specific for S100A9 and/or S100A8 under non-denaturating conditions. As an illustrative example, signal intensity of a detectably labelled immunoglobulin or binding partner, for instance by means of a fluorescent moiety or a moiety generating a fluorescent signal, detecting a target that is found to have an increased molecular weight, may be quantified and used as an indication of complex formation. As a further example, the interaction of S100A9 and S100A8 or of S100A9 and/or a S100A8 with TLR4, optionally of respective functional fragments, may be detected on the basis of based on surface plasmon resonance, for instance using surface plasmon spectroscopy, optical waveguide lightmode spectroscopy or plasmon-waveguide resonance spectroscopy. Surface plasmon resonance, an optoelectronic technique, may be measured label-free or using a label such as a nanoparticle, which may include a metal or a metalloid such as in the form of a quantum dot. In some embodiments a nanoparticle exhibits a surface plasmon resonance at visible wavelengths, possibly including at near-infrared frequencies. Such a nanoparticle may include or consist of a noble metal such as gold or silver, i.e. an element of group 11 of the periodic table of elements (according to the new IUPAC system, group IB according to the old IUPAC system and the CAS system), or an element of group 10 of the periodic table of elements (according to the new IUPAC system, in group VIIIA according to the old IUPAC system and group VIII of the CAS system) such as palladium or platinum. Respective nanoparticles show strong plasmon resonance extinction bands in the visible spectrum, and therefore deep colors reminiscent of molecular dyes. These extinction bands occur if the incident photo frequency is resonant with the collective oscillation of the free (conduction) electrons, also known as the localized surface plasmon resonance (LSPR). LSPR excitation results in wavelength selective absorption with extremely large molar extinction coefficients, efficient Rayleigh scattering and enhanced local electromagnetic fields near the surface of the nanoparticle. A variety of reviews are available providing an introduction into surface plasmon resonance, which is a method well established in the art, as well as its application to sensors (see e.g. Willets, K. A., & Van Duyne, R. P., Annu. Rev. Phys. Chem. (2007) 58, 267-297; Homola, J. et al., Anal Bioanal Chem (2003) 377, 528-539; Schuck, P., Annu. Rev. Biophys. Biomol. Struct. (1997) 26, 541-566; or Hafner, J., Laser Focus World (2006) April, 99-101).

A respective method that includes the measurement of a corresponding complex may in some embodiments include comparing the obtained result to a reference value or to a threshold value. A threshold value may for example be a value set to decide whether a complex is formed or not. A threshold value may also be a value set to decide whether a subject suffers from an inflammatory condition. A threshold value may also be a value set to decide whether a subject suffers from an inflammatory condition that is associated with S100A9 and S100A8.

In some embodiments the method that includes the measurement of a corresponding complex is carried out on a sample from a subject suspected to or known to suffer from an inflammatory condition. A control measurement, in this document also referred to as a reference measurement, may be a measurement that is carried out on a sample from a subject known not to suffer from an inflammatory condition. In some embodiments a respective reference measurement is carried out on a (control) sample from a subject that is age-matched. In some embodiments such a reference measurement is carried out on a sample from the same subject, taken at a previous point of time. In a method as disclosed herein the amount of complex formed, for instance determined in a sample, may be compared to such a reference measurement. In some embodiments the amount of complex determined in a sample is compared to a threshold value. Such a threshold value may in some embodiments be a predetermined threshold value. In some embodiments the threshold value is based the amount of complex determined in a control sample. Generally, a respective control sample may have any condition that varies from the sample used in the main measurement.

In some embodiments the method that includes the measurement of a corresponding complex is carried out in a mixture of the enriched, purified or isolated components of the complex, optionally including a substance suspected to affect the complex formation. Proteins used such as the TLR4 receptor, S100A9 or S100A8 may have been expressed in recombinant form, for example in a suitable host organism. Fragments of the TLR4 receptor, S100A9 or S100A8 may likewise have been obtained by expression in recombinant form. Fragments of the TLR4 receptor, S100A9 or S100A8 may in some embodiments have been synthesized by an established peptide synthesis technique. Such a measurement is generally carried out in an aqueous solution that includes a buffer and/or a salt, such as a calcium salt or a zinc salt. Numerous buffer compounds are used in the art and may be used to carry out the various processes described herein. Examples of buffers include, but are not limited to, solutions of salts of phosphate, carbonate, succinate, citrate, acetate, formate, barbiturate, oxalate, lactate, phthalate, maleate, cacodylate, borate, N-(2-acetamido)-2-amino-ethanesulfonate (also called (ACES), N-(2-hydroxyethyl)-piperazine-N′-2-ethanesulfonic acid (also called HEPES), 4-(2-hydroxyethyl)-1-piperazine-propanesulfonic acid (also called HEPPS), piperazine-1,4-bis(2-ethanesulfonic acid) (also called PIPES), (2-[Tris(hydroxymethyl)-methylamino]-1-ethansulfonic acid (also called TES), 2-cyclohexylamino-ethansulfonic acid (also called CHES) and N-(2-acetamido)-iminodiacetate (also called ADA). Any counter ion may be used in these salts; ammonium, sodium, and potassium may serve as illustrative examples. Further examples of buffers include, but are not limited to, triethanolamine, diethanolamine, ethylamine, triethyl-amine, glycine, glycylglycine, histidine, tris(hydroxymethyl)aminomethane (also called TRIS), bis-(2-hydroxyethyl)-imino-tris(hydroxylmethyl)methane (also called BIS-TRIS), and N-[Tris(hydroxymethyl)-methyl]-glycine (also called TRICINE), to name a few. The buffers may be aqueous solutions of such buffer compounds or solutions in a suitable polar organic solvent. As an illustrative example, a buffer may be deposited in solid form, for example freeze-dried. In such a case the solid buffer, e.g. a powder, may be dissolved in an aqueous phase by merging and or mixing, for instance assisted or performed by means of ultrasound. In such a case the amount of volume of a respective aqueous phase used may for instance be used to obtain the desired final buffer concentration.

In such embodiments, i.e. where a mixture of the enriched, purified or isolated components of the complex are used, a reference measurement may include the use of any condition that varies from the condition of the main measurement. As an illustrative example, where a fragment of the TLR4 receptor, S100A9 and/or S100A8 is used, a reference measurement may encompass the use of the corresponding full length protein(s). In embodiments where a compound is included in the main measurement, which is a compound to be tested for its effect on the respective complex formation, a reference measurement may be a measurement in which this compound is omitted.

In some embodiments a threshold value is a collection of data of a plurality of control samples, which may also be referred to as a reference samples. In such embodiments the threshold value may be set to be a significant difference between the control and the sample from the subject of interest. The term “significant” is used to indicate that the level of increase is of statistical relevance. As an illustrative example a plurality of measurements, including a plurality of samples may have been obtained from the subject of interest. The p value may then be determined A p value of 0.05, 0.02, 0.01 or lower may be taken to indicate a difference. In some embodiments a significant increase is a deviation of a value of a test sample relative to a value of a control sample of about 2 fold or more, including 3 fold or more, such as at least about 5 to about 10 fold or even more.

As indicated above, a predetermined threshold value may in some embodiments be set on the basis of data collected from one or more subjects known not to suffer from a disorder associated with an inflammatory condition. In some embodiments a certain percentile of such data may be used as a threshold value, e.g. a signal intensity measured in a surface plasmon resonance measurement or of an antibody signal detecting a complex formation under non-denaturating conditions (supra). The range of the values of a set of data obtained from samples of subjects or using reference condition in the absence of a test compound, can be divided into 100 equal parts, i.e. percentages of the range can be determined. A percentile represents the value within the respective range below which a certain percent of the data fall, in other words the percentage of the values that are smaller than that value. For example the 95th percentile is the value below which 95 percent of the data are found. In some embodiments a level of proSP-B, or an effective portion thereof, may be regarded as increased or elevated if it is above the 90^{th} percentile, above the 92^{nd} percentile, above the 93^{rd} percentile, above the 94^{th} percentile, above the 95^{th} percentile, above the 96^{th} percentile, above the 97^{th} percentile, above the 98^{th} percentile or above the 99^{th} percentile.

The comparison to a threshold value, which may be a predetermined threshold value, can be carried out manually, semi-automatically or in a fully automated manner. In some embodiments the comparison may be computer assisted. A computer assisted comparison may employ values stored in a database as a reference for comparing an obtained value or a determined amount, for example via a computer implemented algorithm. Likewise, a comparison to a reference measurement may be carried out manually, semi-automatically or in a fully automated manner, including in a computer assisted manner.

In some embodiment the formation of a complex described above may be determined by immobilizing one of the components of the complex on a surface. After contacting the components of the complex with each other and allowing a complex to form, any non-bound components of the complex may be removed, typically by exchanging the medium, e.g. buffer solution encompassing the immobilized complex component. Subsequently the presence of a component of the formed complex, which was not provided in immobilized form, may be determined in order to assess whether a complex has formed, and optionally to which extent such a complex has formed. As an illustrative example it may be intended to determine whether, including to which extent, a complex between a functional fragment of the TLR4 receptor and a S100A9 protein and/or a S100A8 protein has formed. In such an embodiment the fragment of the TLR4 receptor may be immobilized on a surface, for instance on the surface of a well in a multi-well plate. After complex formation and exchange of medium in the well, an immunoglobulin or a proteinaceous binding partner with a binding specificity to S100A9 and/or S100A8 may be used for detection of complex formation. As explained above, an antibody disclosed herein, having a binding specificity to a region on S100A9 and/or S100A8, interacts with S100A9 and

S100A8, respectively, at the site of binding to the TLR4 receptor. Therefore such an antibody can only detect S100A9 and/or S100A8, which is not bound to the TLR4 receptor. Accordingly, for the detection of a S100A9 as well as of a S100A8 protein that is in a complex with the TLR4 receptor, an immunoglobulin or proteinaceous binding partner with a different specificity, i.e. binding to a different site on S100A9 and/or S100A8 will generally be used. Such a binding site on S100A9 is an epitope that differs from the region defined by amino acid positions 63-79 and/or amino acid positions 73-85 of the human protein of Uniprot/Swissprot accession number P06702. A respective binding site on S100A8 is an epitope that differs from the region defined by amino acid positions 55-71 of the human protein of Uniprot/Swissprot accession number P05109 (SEQ ID NO: 78). An antibody of a binding specificity for the region defined by amino acid positions 63-79 and/or amino acid positions 73-85 of the human S100A9 protein may be used in a control measurement to determine whether there is any S100A9 protein left, in which this region is accessible.

Determining the amount of S100A9, S100A8 and/or a TLR4 receptor in a sample can be carried out by way of any suitable technique available. An illustrative example of a suitable technique in this regard is a radiolabel assay such as a Radioimmunoassay (RIA) or an enzyme-immunoassay such as an Enzyme Linked Immunoabsorbent Assay (ELISA), precipitation (particularly immunoprecipitation), a sandwich enzyme immune test, an electro-chemiluminescence sandwich immunoassay (ECLIA), a dissociation-enhanced lanthanide fluoro immuno assay (DELFIA), a scintillation proximity assay (SPA), turbidimetry, nephelometry, latex-enhanced turbidimetry or nephelometry, or a solid phase immune test. Further methods known in the art (such as gel electrophoresis, 2D gel electrophoresis, SDS polyacrylamid gel electrophoresis (SDS-PAGE), Western Blotting, and mass spectrometry), can be used alone or in combination with labelling or other detection methods as described herein. While a RIA is based on the measurement of radioactivity associated with a complex formed between an immunoglobulin or a proteinaceous binding molecule with immunoglobulin-like functions and an antigen, an ELISA is based on the measurement of an enzymatic reaction associated with a complex formed between an immunoglobulin or a proteinaceous binding molecule with immuno-globulin-like functions and an antigen. Typically a radiolabel assay or an enzyme-immunoassay involves one or more separation steps in which a binding partner of e.g. S100A9, S100A8 and/or TLR4 that has not formed a complex with S100A9, S100A8 and/or TLR4 is being removed (cf. above), thereby leaving only binding partner of S100A9, S100A8 and/or TLR4 behind, which has formed a complex with S100A9, S100A8 and/or TLR4. This allows the generation of specific signals originating from the presence of S100A9, S100A8 and/or TLR4.

An ELISA or RIA test can be competitive for measuring the amount of S100A9, S100A8 and/or TLR4, i.e. the amount of antigen. For example, an enzyme labeled antigen is mixed with a test sample containing antigen, which competes for a limited amount of immunoglobulin or a proteinaceous binding molecule with immunoglobulin-like functions. The reacted (bound) antigen is then separated from the free material, and its enzyme activity is estimated by addition of substrate. An alternative method for antigen measurement is the double immunoglobulin/proteinaceous binding molecule sandwich technique. In this modification a solid phase is coated with specific immunoglobulin or a proteinaceous binding molecule with immunoglobulin-like functions. This is then reacted with the sample from the subject that contains the antigen. Then enzyme labelled specific immunoglobulin/proteinaceous binding molecule is added, followed by the enzyme substrate. The ‘antigen’ in the test sample is thereby ‘captured’ and immobilized on to the sensitized solid phase where it can itself then immobilize the enzyme labelled immunoglobulin/proteinaceous binding molecule. This technique is analogous to the immunoradiometric assays.

In an indirect ELISA method, an antigen is immobilized by passive adsorption on to the solid phase. A test serum may then be incubated with the solid phase and any immunoglobulin in the test serum forms a complex with the antigen on the solid phase. Similarly a solution of a proteinaceous binding molecule with immunoglobulin-like functions may be incubated with the solid phase to allow the formation of a complex between the antigen on the solid phase and the proteinaceous binding molecule. After washing to remove unreacted serum components an anti-immunoglobulin immunoglobulin anti-proteinaceous binding molecule immunoglobulin, linked to an enzyme is contacted with the solid phase and incubated. Where the second reagent is selected to be a proteinaceous binding molecule with immunoglobulin-like functions, a respective proteinaceous binding molecule that specifically binds to the proteinaceous binding molecule or the immunoglobulin directed against the antigen is used. A complex of the second proteinaceous binding molecule or immunoglobulin and the first proteinaceous binding molecule or immunoglobulin, bound to the antigen, is formed. Washing again removes unreacted material. In the case of RIA radioactivity signals are being detected. In the case of ELISA the enzyme substrate is added. Its colour change will be a measure of the amount of the immobilized complex involving the antigen, which is proportional to the antibody level in the test sample.

In another embodiment the immunoglobulin or the proteinaceous binding molecule with immunoglobulin-like functions may be immobilized onto a surface, such as the surface of a polymer bead (supra), or coated onto the surface of a device such as a polymer plate or a glass plate. Such an embodiment may be employed in combination with the measurement of the formation of a complex described above. An immunoglobulin or proteinaceous binding molecule with a binding specificity to S100A9, S100A8 and/or TLR4 may be employed to immobilize the respective target of antibody binding to the surface. A complex may then be allowed to form after providing the remaining components of the complex, optionally also providing a compound to be tested for affecting complex formation. Thereafter the formation of the complex may be detected using a suitable immunoglobulin or proteinaceous binding molecule. By immobilisation, in a detection technique such as ELISA, the immune complexes can easily be separated from other components present by simply washing the surface, e.g. the beads or plate. This is the most common method currently used in the art and is referred to as solid phase RIA or ELISA. This embodiment may be particularly useful for determining the amount of S100A9, S100A8 and/or TLR4. On a general basis, in any embodiment of a radiolabel assay or of an enzyme-immunoassay passive adsorption to the solid phase can be used in the first step. Adsorption of other reagents can be prevented by inclusion of wetting agents in all the subsequent washing and incubation steps. It may be advantageous to perform washing to prevent carry-over of reagents from one step to the next.

Various other modifications of ELISA have been used in the art. For example, a system where the second proteinaceous binding molecule or immunoglobulin used in the double antibody sandwich method is from a different species, and this is then reacted with an anti-immunoglobulin enzyme conjugate or an anti-proteinaceous binding molecule enzyme conjugate. This technique comes with the potential advantage that it avoids the labeling of the specific immunoglobulin or proteinaceous binding molecule, which may be in short supply and of low potency. This same technique can be used to assay immunoglobulin or proteinaceous binding molecule where only an impure antigen is available; the specific reactive antigens are selected by the antibody immobilized on the solid phase.

In another example of an ELISA assay for antigen, a surface, a specific antigen is immobilized on a surface, e.g. a plate used, and the surface is then incubated with a mixture of reference immunoglobulins or proteinaceous binding molecules and a test sample. If there is no antigen in the test sample the reference immunoglobulin or proteinaceous binding molecule becomes fixed to an antigen sensitized surface. If there is antigen in the test solution this combines with the reference immunoglobulin or proteinaceous binding molecule, which cannot then react with the sensitized solid phase. The amount of immunoglobulin/proteinaceous binding molecule attached is then indicated by an enzyme labeled anti-globulin/anti-binding molecule conjugate and enzyme substrate. The amount of inhibition of substrate degradation in the test sample (as compared with the reference system) is proportional to the amount of antigen in the test system.

In some embodiments the amount of S100A9 and/or a S100A8, or the proportion of S100A9, in which the region corresponding to amino acid positions 63-79 and/or 73-85 of the human protein S100A9, and/or the region corresponding to amino acid positions 55-71 of the human protein S100A8 are not accessible, determined in or from a sample of a subject can be compared to a single control sample or a plurality of control samples, such as a sample from a control subject, in any suitable manner. As an illustrative example, the level of heterodimers and or heterotetramers of S100A9 and S100A8 in a control sample can be characterized by an average (mean) value coupled with a standard deviation value, for example at a given time point. In some embodiments the level of heterodimers and or heterotetramers of S100A9 and S100A8 in a subject may be considered increased or decreased when it is one standard deviation or more higher or lower than the average value of the corresponding heterodimer/tetramer determined in one or more control samples. In some embodiments the determined level of heterodimer/tetramer is regarded as increased or decreased where the obtained value is about 1.5 standard deviations higher or lower, including about two, about three, about four or more standard deviations higher or lower than the average value determined in a control sample. In some embodiments the determined amount of heterodimer/tetramer is regarded as different where the obtained value is about 1.2 times or more higher or lower, including about 1.5 times, about two fold, about 2.5-fold, about three fold, about 3.5 fold, about 4-fold, about 5-fold or more higher or lower than the protein level determined in a control sample. In some embodiments the determined level of heterodimer/tetramer is regarded as increased where the obtained value is about 0.8-fold or less, including about 70%, about 60%, about 50%, about 40%, about 30%, about 25%, about 20% or lower than the amount of heterodimers and or heterotetramers of S100A9 and S100A8 determined in a control sample.

The compound or combination described herein, including an immunoglobulin or a proteinaceous binding partner, as well as a compound or combination identified by a method as disclosed herein, can be administered to a cell, an animal or a human patient per se, or in pharmaceutical compositions where they are mixed with other active ingredients, as in combination therapy, or suitable carriers or excipient(s), including stabilizers. Such carriers, excipients or stabilizers are usually pharmaceutically acceptable in that they are nontoxic to the cell or mammal being exposed thereto at the dosages and concentrations employed. Often the physiologically acceptable carrier is an aqueous pH buffered solution. Examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN®, polyethylene glycol (PEG), and PLURONICS®. Exemplary routes include, but are not limited to, oral, transdermal, and parenteral delivery.

Suitable routes of administration may, for example, include depot, oral, rectal, transmucosal, or intestinal administration; parenteral delivery, including intramuscular, subcutaneous, intravenous, intramedullary injections, as well as intrathecal, direct intraventricular, intraperitoneal, intranasal, or intraocular injections.

Alternately, one may administer the compound or combination in a local rather than systemic manner, for example, via injection of the compound or combination directly into a tissue, often in a depot or sustained release formulation.

Furthermore, one may administer the drug in a targeted drug delivery system, for example, in a liposome coated with a tumour-specific antibody. The liposomes will be targeted to and taken up selectively by the tumour.

A pharmaceutical composition disclosed herein includes a compound or combination as defined above. Such a pharmaceutical composition may be manufactured in a manner that is itself known, e.g., by means of conventional mixing, dissolving, granulating, dragee-making, levigating, emulsifying, encapsulating, entrapping or lyophilizing processes.

Pharmaceutical compositions for use in accordance with the present invention thus may be formulated in conventional manner using one or more physiologically acceptable carriers including excipients and auxiliaries that facilitate processing of the active compound or combination into preparations that can be used pharmaceutically. Proper formulation is dependent upon the route of administration chosen.

For injection, the agents disclosed herein may be formulated in aqueous solutions, for instance in physiologically compatible buffers such as Hanks's solution, Ringer's solution, or physiological saline buffer. For transmucosal administration, penetrants appropriate to the barrier to be permeated are used in the formulation. Such penetrants are generally known in the art.

For oral administration, the compound or combination can be formulated readily by combining the compound or combination with pharmaceutically acceptable carriers well known in the art. Such carriers enable the compound or combination disclosed herein to be formulated as a tablet, pills, dragee, capsule, liquid, gel, syrup, slurry or suspension, for oral ingestion by a patient to be treated.

Pharmaceutical preparations for oral use can be obtained by adding a solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP).

If desired, disintegrating agents may be added, such as the cross-linked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound or combination doses.

Pharmaceutical preparations that can be used orally include push-fit capsules made of gelatine, as well as soft, sealed capsules made of gelatine and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compound or combination may be dissolved or suspended in suitable liquids, such as fatty oils, liquid paraffin, or liquid polyethylene glycols. In addition, stabilizers may be added. All formulations for oral administration should be in dosages suitable for such administration. For buccal administration, the compositions may take the form of tablets or lozenges formulated in conventional manner.

For administration by inhalation, the compound or combination for use as disclosed herein is conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g. gelatine for use in an inhaler or insufflator may be formulated containing a powder mix of the compound or combination and a suitable powder base such as lactose or starch.

The compound or combination may be formulated for parenteral administration by injection, e.g., by bolus injection or continuous infusion. Formulations for injection may be presented in unit dosage form, e.g., in ampules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compound or combination in water-soluble form. Additionally, a suspension of the active compound or combination may be prepared as an appropriate oily injection suspension. Suitable lipophilic solvents or vehicles include fatty oils such as sesame oil, or synthetic fatty acid esters, such as ethyl oleate or triglycerides, or liposomes. Aqueous injection suspensions may contain substances that increase the viscosity of the suspension, such as sodium carboxymethyl cellulose, sorbitol, or dextran. Optionally, the suspension may also contain suitable stabilizers or agents that increase the solubility of the compound or combination to allow for the preparation of highly concentrated solutions.

Alternatively, the active ingredient may be in powder form for constitution with a suitable vehicle, e.g., sterile pyrogen-free water, before use. The compound or combination may also be formulated in rectal compositions such as suppositories or retention enemas, e.g., containing conventional suppository bases such as cocoa butter or other glycerides.

In addition to the formulations described previously, the compound or combination may also be formulated as a depot preparation. Such long acting formulations may be administered by implantation (for example subcutaneously or intramuscularly) or by intramuscular injection. Thus, for example, the compound or combination may be formulated with suitable polymeric or hydrophobic materials (for example, as an emulsion in an acceptable oil) or ion exchange resins, or as sparingly soluble derivatives, for example, as a sparingly soluble salt.

A pharmaceutical carrier for a hydrophobic compound or combination disclosed herein is a co-solvent system including benzyl alcohol, a non-polar surfactant, a water-miscible organic polymer, and an aqueous phase. The co-solvent system may be the VPD co-solvent system. VPD is a solution of 3% w/v benzyl alcohol, 8% w/v of the non-polar surfactant polysorbate 80, and 65% w/v polyethylene glycol 300, made up to volume in absolute ethanol. The VPD co-solvent system (VPD: D5W) consists of VPD diluted 1:1 with a 5% dextrose in water solution.

This co-solvent system dissolves hydrophobic compound or combination well, and itself produces low toxicity upon systemic administration. Naturally, the proportions of a co-solvent system may be varied considerably without destroying its solubility and toxicity characteristics.

Furthermore, the identity of the co-solvent components may be varied: for example, other low-toxicity non-polar surfactants may be used instead of polysorbate 80; the fraction size of polyethylene glycol may be varied; other biocompatible polymers may replace polyethylene glycol, e.g. polyvinyl pyrrolidone; and other sugars or polysaccharides may substitute for dextrose.

Other delivery systems for hydrophobic pharmaceutical compounds may also be employed. Liposomes and emulsions are well known examples of delivery vehicles or carriers for hydrophobic drugs. Certain organic solvents such as dimethylsulfoxide also may be employed, although usually at the cost of greater toxicity. Additionally, the compound or combination may be delivered using a sustained-release system, such as semipermeable matrices of solid hydrophobic polymers containing the therapeutic agent. Various types of sustained-release materials have been established and are well known by those skilled in the art. Sustained-release capsules may, depending on their chemical nature, release the compound or combination for a few weeks up to over 100 days. Depending on the chemical nature and the biological stability of the therapeutic reagent, additional strategies for protein stabilization may be employed.

The pharmaceutical compositions also may include suitable solid or gel phase carriers or excipients.

Examples of such carriers or excipients include but are not limited to calcium carbonate, calcium phosphate, various sugars, starches, cellulose derivatives, gelatine, and polymers such as polyethylene glycols.

Many of the compounds that may be used in the context of the invention may be provided as salts with pharmaceutically compatible counter-ions. Pharmaceutically compatible salts may be formed with many acids, including but not limited to hydrochloric, sulfuric, acetic, lactic, tartaric, malic, succinic, etc. Salts tend to be more soluble in aqueous or other protonic solvents that are the corresponding free base forms.

Pharmaceutical compositions suitable for use in the context of the present invention include compositions where the active ingredients are contained in an amount effective to achieve its intended purpose. More specifically, a therapeutically effective amount means an amount of compound effective to prevent, alleviate or ameliorate symptoms of disease or prolong the survival of the subject being treated. Determination of a therapeutically effective amount is well within the capability of those skilled in the art, especially in light of the detailed disclosure provided in this document.

For any compound used in the methods disclosed herein, the therapeutically effective dose can be estimated initially from cell culture assays. For example, a dose can be formulated in animal models to achieve a circulating concentration range that includes the IC50 as determined in cell culture (i.e., the concentration of the test compound which achieves a half-maximal inhibition of the kinase activity). Such information can be used to more accurately determine useful doses in humans.

Toxicity and therapeutic efficacy of the compound or combination described herein can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio between LD₅₀ and ED₅₀. It may be desired to use a compound or combination that exhibit high therapeutic indices. The data obtained from these cell culture assays and animal studies can be used in formulating a range of dosage for use in humans. The dosage of such compound or combination lies preferably within a range of circulating concentrations that include the ED₅₀ with little or no toxicity. The dosage may vary within this range depending upon the dosage form employed and the route of administration utilized. The exact formulation, route of administration and dosage can be chosen by the individual physician in view of the patient's condition.

Dosage amount and interval may be adjusted individually to provide plasma levels of the active moiety, which are sufficient to maintain the kinase modulating effects, or minimal effective concentration (MEC). The MEC will vary for each compound or combination but can be estimated from in vitro data; e.g., the concentration necessary to achieve 50-90% inhibition of the kinase. Dosages necessary to achieve the MEC will depend on individual characteristics and route of administration. However, HPLC assays or bioassays can be used to determine plasma concentrations.

Dosage intervals can also be determined using MEC value. Compounds should be administered using a regimen that maintains plasma levels above the MEC for 10-90% of the time, for example from about 30 to about 90%, such as from about 50 to about 90%. In cases of local administration or selective uptake, the effective local concentration of the drug may not be related to plasma concentration. The amount of composition administered will, of course, be dependent on the subject being treated, on the subject's weight, the severity of the affliction, the manner of administration and the judgment of the prescribing physician.

The compositions may, if desired, be presented in a pack or dispenser device, which may contain one or more unit dosage forms containing the active ingredient. The pack may for instance include metal or plastic foil, such as a blister pack. The pack or dispenser device may be accompanied by instructions for administration. The pack or dispenser may also be accompanied with a notice associated with the container in a form prescribed by a governmental agency regulating the manufacture, use, or sale of pharmaceuticals, which notice is reflective of approval by the agency of the form of the compound for human or veterinary administration. Such notice, for example, may be the labelling approved by the U. S. Food and Drug Administration or other government agency for prescription drugs, or the approved product insert.

Compositions disclosed herein formulated in a compatible pharmaceutical carrier may also be prepared, placed in an appropriate container, and labelled for treatment of an indicated condition. Suitable conditions indicated on the label may include, for example, treatment of cancer.

As explained above, the present invention inter alia encompasses the diagnostic, prognostic, and therapeutic use of an immunoglobulin or proteinaceous binding molecule capable of binding to and modulating the activity of a S100A8 protein and/or a S100A9 protein. Based on the inventors' findings provided are also methods of identifying a compound that is capable of preventing, inhibiting, arresting or reversing a condition associated with inflammation. Some of these methods are in vivo or ex vivo methods. Some of the methods are in-vitro methods of identifying a respective peptide, peptidomimetic or combination.

The listing or discussion of a previously published document in this specification should not necessarily be taken as an acknowledgement that the document is part of the state of the art or is common general knowledge.

The invention illustratively described herein may suitably be practiced in the absence of any element or elements, limitation or limitations, not specifically disclosed herein. Thus, for example, the terms “comprising”, “including,” containing”, etc. shall be read expansively and without limitation. Singular forms such as “a”, “an” or “the” include plural references unless the context clearly indicates otherwise. Unless otherwise indicated, the term “at least” preceding a series of elements is to be understood to refer to every element in the series. The terms “at least one” and “at least one of” include for example, one, two, three, four, or five or more elements. Slight variations above and below the stated ranges can be used to achieve substantially the same results as values within the ranges. Also, unless indicated otherwise, the disclosure of the ranges is intended as a continuous range including every value between the minimum and maximum values.

Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by exemplary embodiments and optional features, modification and variation of the inventions embodied therein herein disclosed may be resorted to by those skilled in the art, and that such modifications and variations are considered to be within the scope of this invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the appending claims. In addition, where features or aspects of the invention are described in terms of Markush groups, those skilled in the art will recognize that the invention is also thereby described in terms of any individual member or subgroup of members of the Markush group.

In order that the invention may be readily understood and put into practical effect, particular embodiments will now be described by way of the following non-limiting examples.

### EXAMPLES

Using standard techniques known in the art, the inventors expressed the individual human proteins S100A8 and S100A9 in recombinant form, and purified them. After generating homodimers and heterodimers, they analysed the properties of the complexes. FIG. 1 illustrates the stimulation of human monocytes for four hours with the indicated concentrations of (A) recombinant human S100A8, recombinant human S100A9 or human S100A8/S100A9, and (B) recombinant human S100A8/S100A9, recombinant human S100A8/S100A9 (N69A) or S100A8/S100A9 (E78A). TNFα released into the culture medium was quantified by means of ELISA.

While the homodimers showed activating properties on monocytes, the heterotetrameric complex of s100A8 and S100A9 did not show activating properties that would be comparable to the individual components (FIG. 1A). By means of site directed mutagenesis, preventing the formation of (S100A8/S100A9)₂ tetramers, the inventors found that the formation of tetramers blocks certain amino acids that are important for binding to TLR4.

Mutating specific amino acids in the second calcium binding EF hand in S100A9, namely N69 and E78, causes an inhibition of tetramer formation. Further, this mutation leads to an activation of monocytes that is comparable to the activation caused by homodimers (FIG. 1B). Accordingly, the activity of S100A8 and S100A9 is controlled by their oligomerisation state.

Expression and Purification of S100A8 and S100A9 Proteins.

For the expression of recombinant (rec) proteins without additional peptide sequences, the cDNAs from wt S100A8, wt S100A9 and the S100A9 EF-hand mutants were cloned into the pET11/20 vector [50-NdeI; 30-BamHI]. Expression and isolation of the gene products was achieved in E. coli strain BL21 (DE3). Bacteria were grown at 37° C. in 2×YT for 24 h. Afterwards bacteria were harvested, lysed and the inclusion bodies (IB) prepared. The IB pellet was dissolved in 8 M urea buffer and to establish proper refolding samples were adjusted to pH 2.0-2.5 first by adding hydrochloric acid. After 60 min incubation at room temperature, samples were stepwise dialyzed to get adapted to pH 7.4 for refolding in the presence of 2 mM DTT. After centrifugation (10 min, 60,000 g, 4° C.) to pellet aggregated material, samples were further dialyzed and applied to anion exchange column and gel filtration chromatography. To prepare heterodimeric complexes the recombinant proteins were mixed 1:1 in equimolar concentrations first. Samples were stored as stock solutions at −20° C. Correct refolding and complex formation was assessed by SDS-PAGE, CD spectroscopy, MALDI-MS and ESI-MS.

The maximal endotoxin contamination in the S100 preparations was determined by Limulus amoebocyte lysate (LAL) assay (BioWhitaker, Walkersville, Md.) and was lower than 1 pg LPS/μg S100 protein or could not be detected in the different batches. In addition PolymyxinB (50 μg/ml; Sigma) was added to S100A8 in control experiments to exclude stimulatory effects due to LPS contamination.

Preparation and Stimulation of Monocytes.

Monocytes were isolated from human buffy coats by Ficoll-Paque and subsequent Percoll density centrifugation (Pharmacia, Freiburg, Germany) Cells were cultured in Teflon bags (Biofolie 25; Heraeus Instruments, Hanau, Germany) using McCoy's 5a medium supplemented with 15% fetal calf serum for 1 day before stimulation. Monocytes were incubated for 4 hours with different dosis of hS100A8, hS100A9, hS100A8/S100A9 or the modified proteins as indicated in the figures and TNF-α concentrations in supernatants were determined by ELISA (OptEIA, BD Biosciences, Germany).

Determination of Cytokine Concentrations.

Release of cytokine TNF-α was measured in the culture supernatants by ELISA (OptEIA, BD Biosciences).

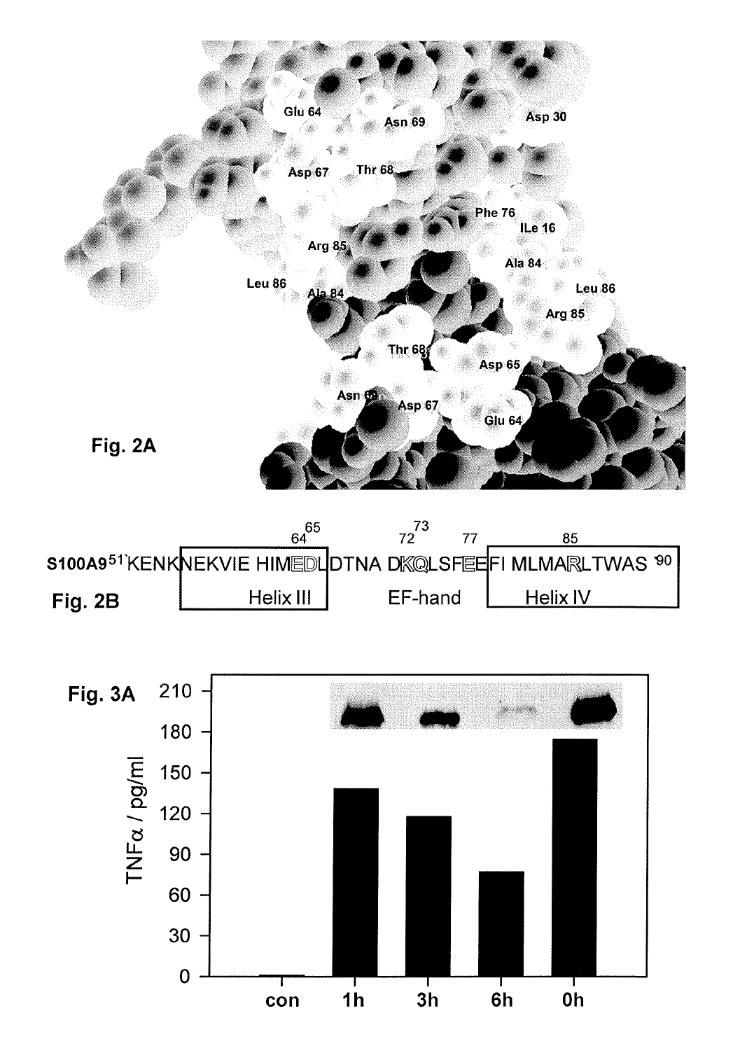

Using a computer-assisted approach based on the 3D structures of homodimer, heterodimer and heterotetramer, which are known in the art, the inventors identified those amino acids of S100A9 that are freely accessible in the homodimeric form and in the heterodimer of S100A8 and S100A9, but that are blocked in the heterotetrameric form (S100A8/S100A9)₂. They found that predominantly amino acids located in the C-terminal EF hand, also termed EF hand II, are involved (FIG. 2A). Certain of these amino acids, being amino acids not concurrently involved in calcium binding, were subsequently selected for mutation studies (FIG. 2B, namely the amino acids of positions of the human protein S100A9 of the Uniprot/Swissprot accession number P06702 (version 147 as of 5 Sep. 2012, SEQ ID NO: 77).

Computer Assisted Ligand/Receptor Interaction Studies:

PDB files of S100A8/A9 tetramer (PDB ID: 1XK4), S100A9 (PDB ID: 1IRJ) and S100A8 (PDB id: 1MR8) were retrieved from RSCB PDB website. The S100A8/A9 pdb file was modified so that it contained only the E and G chains resembling the heterodimer. The modified S100A8/A9 file was analysed using computer modelling programs as Autodock (3D Computer modelling program), Pymol and Swiss-PDBviewer to analyse the aminoacids which are free in the heterodimer or S100A9 homodimer but buried in the tetramer (interface analysis). We concentrated our analyses on the identification of aminoacids that in addition are not involved in Ca++ binding and sterically free for binding to TLR4 Amino acids in S100A9 (positions 64, 65, 72, 73, 77 and 85) were chosen for mutation studies.

Mutations at amino acid positions 64 (glutamic acid), 65 (aspartic acid), 73 (glutamine) and 77 (glutamic acid) caused a loss of function also for the S100A9 homodimer. Mutations at amino acid positions 72 (lysine) and 85 (arginine) caused hardly any effect. These studies with purified mutant proteins show that EF hand II is indeed responsible for the binding to and the activation of TLR4.

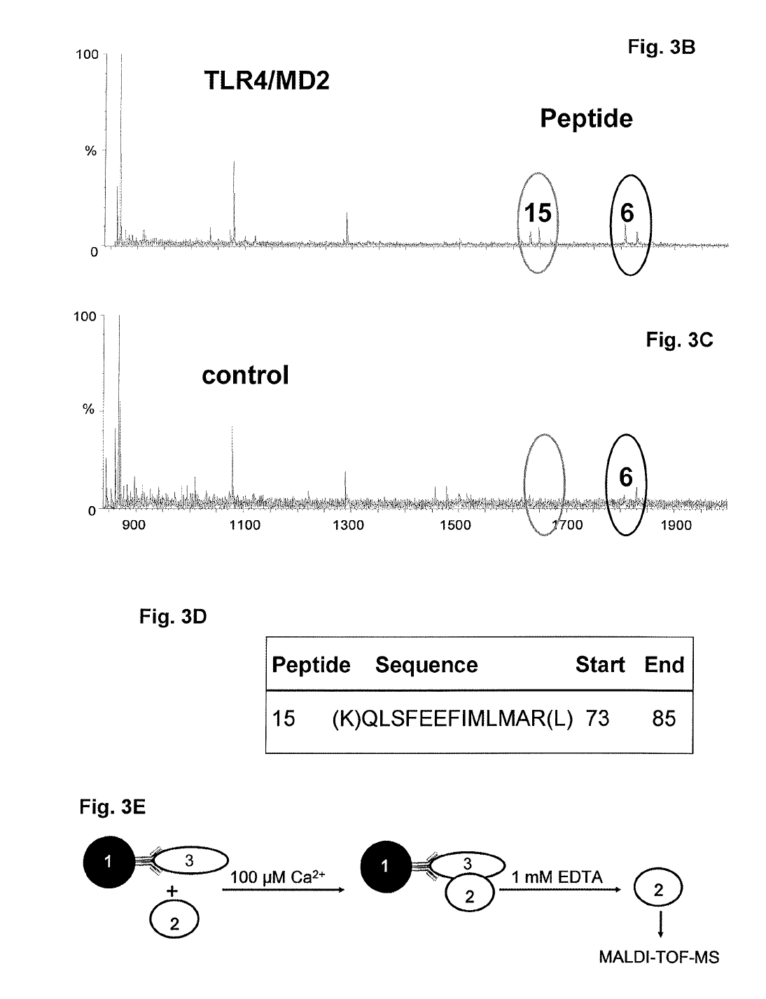

In a methodically independent parallel approach, S100A9 was partially digested with trypsin. The obtained peptide fragments were examined with regard to their capability of still activating monocytes. It was found that one or more fragments of S100A9 were apparently still able to activate monocytes, even if as good as no intact S100A9 protein molecule was detectable any more (FIG. 3A). The particular peptide was isolated by means of sepharose beads, to which TLR4/MD2 had been coupled. The peptide was analysed by mass spectrometry. A peptide was identified, which consisted of the amino acid sequence from positions 73 to 85 of S100A9. The identified peptide coincided very well with the results of the computer-based simulation approach and with the mutation studies.

Tryptic Digestion of Human S100A9 Homodimer:

Immobilized TPCK Trypsin (25 μl of settled gel, Pierce, Rockford) was used to digest 30 μg of human S100A9 at 37° C. for different time points as indicated in the figure and subsequently samples were centrifuged (5 mM, 400×g) using a resin separator to remove trypsinbeads. Aliquots were taken from the centrifugate and either analysed by SDS-PAGE/WesternBlot or to stimulate human monocytes for 4 hours. TNF-a concentrations in supernatants of stimulated monocytes were determined by ELISA (OptEIA, BD Biosciences, Germany).

Western Blot Analysis:

Trypsin digested peptidic fragments of S100A9 were separated on SDS-polyacrylamide gels and transferred to nitrocellulose membranes (Schleicher and Schuell). Membranes were blocked with 5% skim milk powder and subsequently probed with the primary antibody a-S100A9 (rabbit, polyclonal, 1 μg/ml) over night at 4° C. Afterwards bound primary antibody was detected with HRP-conjugated secondary antibody (goat anti rabbit-HRP) and developed with enhanced chemoluminescence system (ECL).

Immunoprecipitation Studies to Identify TLR4/MD2 Binding Peptides:

Anti-His antibody (5 μL, 0.5 mg/mL, Invivogen) and his-tagged rhTLR4/MD2 (5 μL, 1 mg/mL, carrier free, R&D SYSTEMS) were mixed and coupled to Protein A/G Agarose (50 μl, Pierce, Thermo Scientific). Trypsin digested peptides of S100A9 were added for 3 h at 4° C. in the presence of 1 mM Calcium. After washing of the beads in HBS/1 mM Ca-buffer for three times bound peptidic fragments were eluted by addition of 10 mM TRIS/2 mM EDTA-buffer and analysed by ESI-QIT- and MALDI-TOF-mass spectrometry. Identical experiments were performed to analyze the binding of the chemical synthesized peptides of S100A9 (aa63-79), S100A8 (aa55-71) and the corresponding control peptides aa63-79 AS and aa55-71 A3. A schematic of the immunoprecipitation test is shown in FIG. 3E.

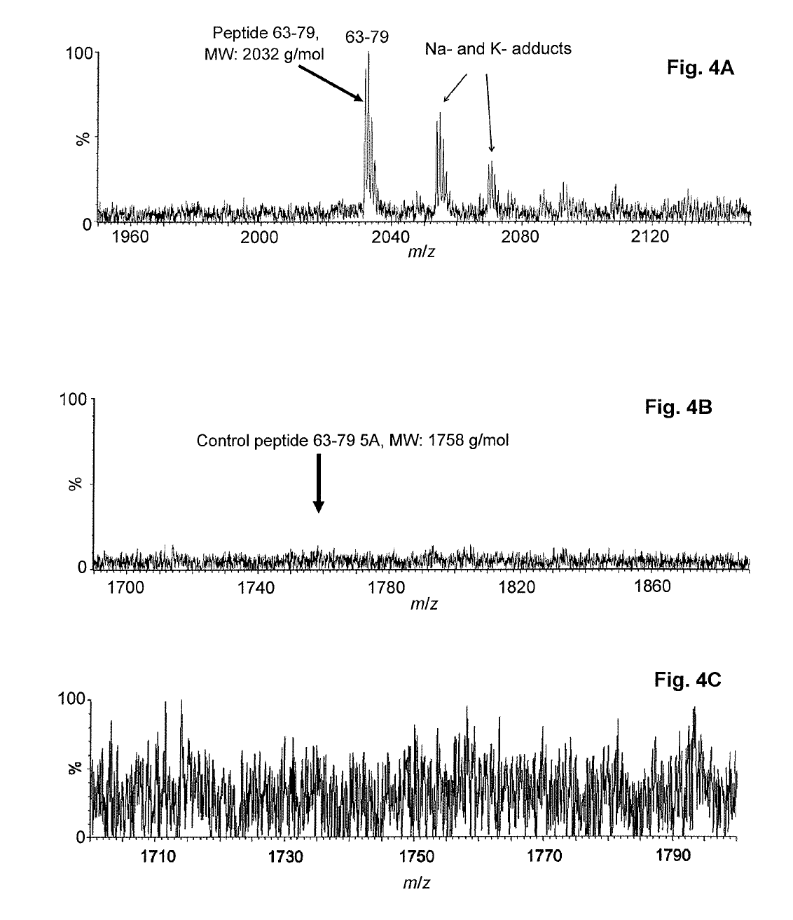

In yet a further approach the inventors examined a synthetic peptide with a sequence corresponding to amino acid positions 63-79, i.e. the complete C-terminal EF hand (MEDLDTNADKQLSFEEF, molecular weight: 2032 g/mol) of S100A9 with regard to its binding to TLR4/MD2. A peptide with the sequence of amino acid positions 63-79 (63-79 5A, molecular weight: 1758 g/mol) of S100A9 served as a control, in which the four amino acids identified as most likely important for binding to TLR4/MD2 (E64A, D65A, Q73A and E77A, nomenclature of S100A9 maintained), and in addition amino acid K72A, had been exchanged to alanine. A comparison of FIG. 4A and FIG. 4B shows clearly that only the non-mutant peptide (63-79) is able to bind to TLR4/MD2. In contrast thereto, for the peptide with 5 mutant amino acids (63-79 A5) no binding could be detected, even in an enlargement on the Y axis (peak at 1758 m/z).

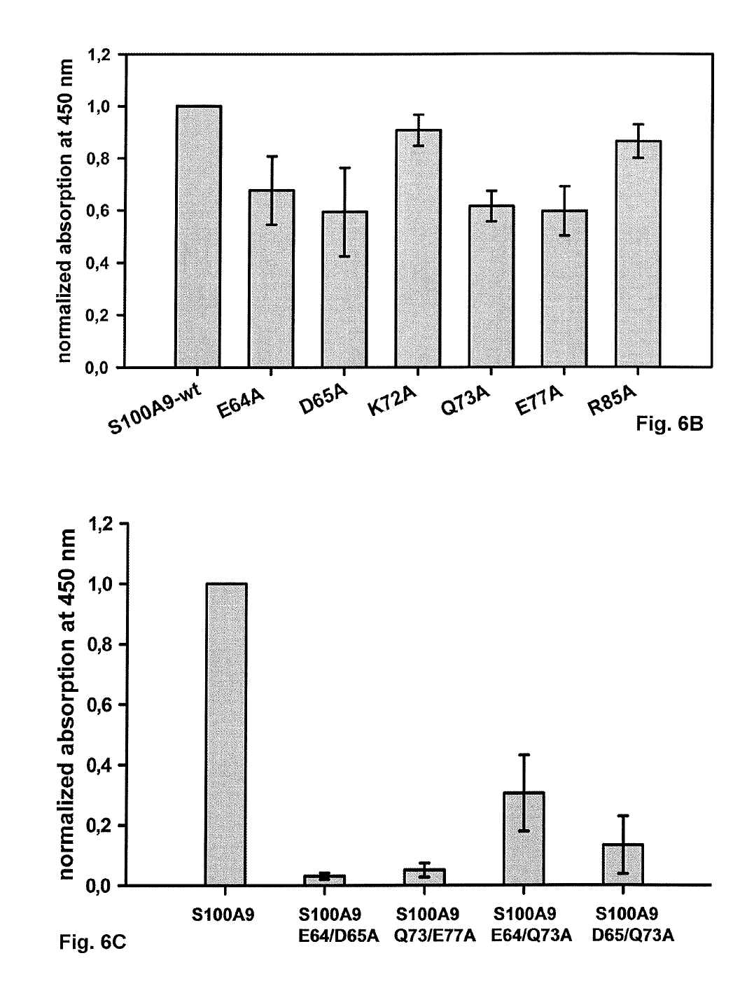

In a parallel approach the inventors used mutants of S100A9, which contained mutations in the region supposedly involved in binding to TLR4/MD2. These S100A9 mutants were used in the form of purified proteins and contained one or two mutated amino acids, in that one or two amino acids in the region of positions 63-79 were exchanged for an alanine. As can be taken from FIG. 6B, the mutated proteins S100A9E64A, S100A9D65A, S100A9Q73A, and S100A9E77A showed a weaker binding to the receptor when compared to non-mutated protein (S100A9 wt). The mutated proteins S100A9K72A and S100A9R85A showed a binding that was not significantly different from the wild type protein S100A9 (FIG. 6B). Mutated proteins of S100A9 that contained an amino acid exchange at two positions when compared to the wild type protein showed an almost complete loss of binding to the receptor. This observation further proves the importance of this region of S100A9 and of amino acids E54, D65, Q73 and E77 for receptor interaction.

Binding of S100A9-Wt and Mutant Proteins to TLR4/MD2:

Binding of S100A9 proteins to TLR4/MD2 was analysed by a modified S100A9-ELISA. Briefly, TLR4/MD2 was coupled to the wells of a 96-well plate and served as capturing molecule. After blocking of the unspecific binding sites by PBS/5% skim milk powder plates were washed three times. S100A9-wt or mutant S100A9 proteins were added at a concentration of 2 μg/ml each in the presence and absence of 100 μM Calcium and incubated for two hours at room temperature. Unbound S100A9 was removed by washing the plates for three times followed by the addition of a primary anti-S100A9-antibody (1 μg/ml, polyclonal, rabbit). After a washing step the secondary anti-rabbit-IgG-antibody coupled to HRP (1 μg/ml from Cell Signalling) was added. TMB was used as substrate for HRP to quantify binding by absorbance readings at 450 nm in an ELISA reader (Anthos Mirkosysteme).

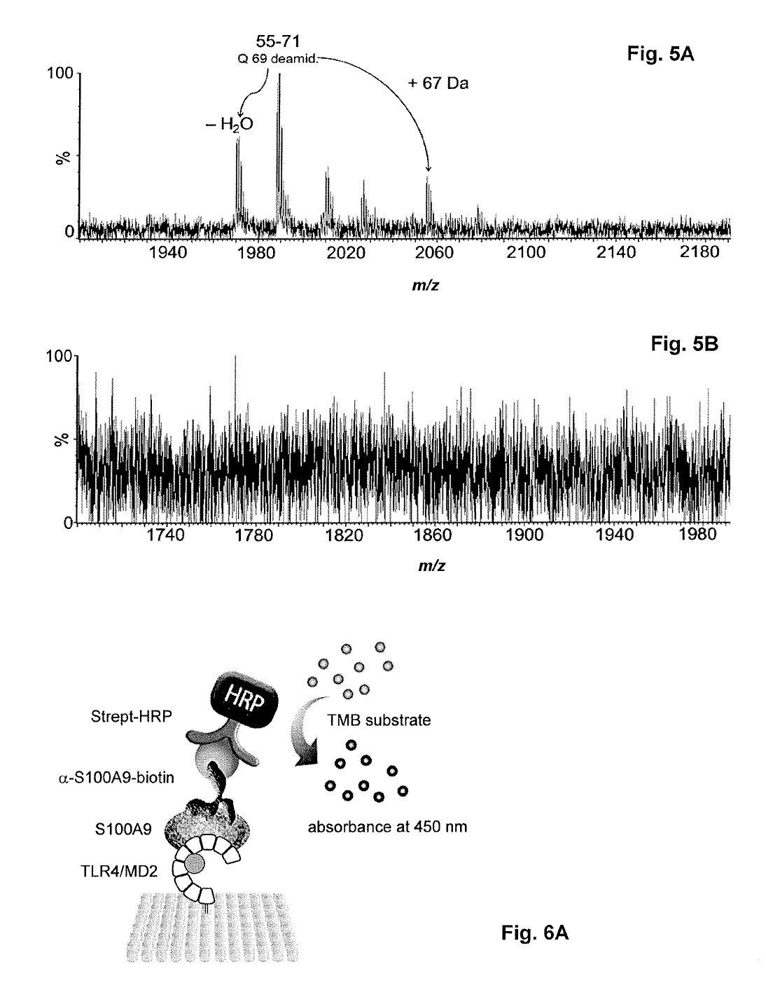

Finally, the inventors analysed a synthetic peptide, having the amino acid sequence of positions 55-71 of human S100A8 (Uniprot/Swissprot accession number P05109, version 138 as of 5 Sep. 2012, SEQ ID NO: 78), i.e. the complete C-terminal EF hand (FKELDINTDGAVNFQEF, molecular weight: 1990 g/mol) with regard to its binding to TLR4/MD2. Again, a peptide with the sequence of amino acid positions 55-71 (55-71 3A, molecular weight: 1815 g/mol) of S100A8 served as a control, in which those amino acids identified as most likely important for binding to TLR4/MD2, analogously to S100A9, were exchanged to alanine. Although the purity of the peptide was not optimal, a comparison of FIG. 5A and FIG. 5B shows that only the non-mutant peptide 55-71 (FIG. 5A) is able to bind to TLR4/MD2. For the peptide with 3 mutant amino acids 55-71A3, however, no binding could be detected, even in an enlargement on the Y axis (Peak with 1815 m/z).

In summary, these data show that the C-terminal calcium binding hands, corresponding to amino acid positions 63-79 of human S100A9 (MEDLDTNADKQLSFEEF, molecular weight: 2032 g/mol) and amino acid positions 55-71 of S100A8 (FKELDINTDGAVNFQEF, molecular weight: 1990 g/mol) mediate the interaction of the respective protein with TLR4.

What is claimed is:

1. An immunoglobulin or proteinaceous binding partner having a binding specificity to an epitope of a vertebrate S100A9 protein, wherein the epitope has an amino acid sequence of a region corresponding to (i) the amino acid sequence ranging from amino acid position 63 to amino acid position 79 of the human protein S100A9 of Uniprot/Swissprot accession no. P06702 (SEQ ID NO: 77) or (ii) the amino acid sequence ranging from amino acid position 73 to amino acid position 85 of the human protein S100A9 of Uniprot/Swissprot accession no. P06702 (SEQ ID NO: 77).

2. The immunoglobulin or proteinaceous binding partner of claim 1, wherein the amino acid sequence is one of the sequences MEDLDTNADKQLSFEEF (SEQ ID NO: 1), MEDLDTNEDKQLSFEEF (SEQ ID NO: 14), MEDLDTNVDKQLSFEEF (SEQ ID NO: 15), MEDLDTNLDKQLSFEEF (SEQ ID NO: 16), MEDLDTNGDKQLNFEEF (SEQ ID NO: 17), LEDLDTNADKQLTFEEF (SEQ ID NO: 18), LEDLDTNVDKQLS FEEF (SEQ ID NO: 19), LEDLDTNEDKQLSFEEF (SEQ ID NO: 20), MEDLDTN GDKELNFEEF (SEQ ID NO: 21), MEDLDTNEDKELSFEEY (SEQ ID NO: 22), LEDLDTNGDKQLNFEEF (SEQ ID NO: 23), MEDLDTNQDNQLSFEEC (SEQ ID NO: 24), MEDLDTNLDQQLSFEEL (SEQ ID NO: 25), MQDLDTNQDQQLSFEEV (SEQ ID NO: 26), MEDLDTNQDKQLSFEEF (SEQ ID NO: 27), MQELDTNQ NGQVDFKEF (SEQ ID NO: 28), FEETDLNKDKELTFEEF (SEQ ID NO: 29), QLSFEEFIMLMAR (SEQ ID NO: 3), QLSFEEFIVLMAR (SEQ ID NO: 30), QLSFEEFIMLVAR (SEQ ID NO: 31), QLTFEEFIMLMGR (SEQ ID NO: 32), QLSFEEFIMLVIR (SEQ ID NO: 33), QLSFEEFIILVAR (SEQ ID NO: 34), QLSFEELTMLLAR (SEQ ID NO: 35), QLSFEEVIMLFAR (SEQ ID NO: 36), QLSFEEFSILMAK (SEQ ID NO: 37), QLSFEEFSMLVAK (SEQ ID NO: 38), QLSFEECMMLMAK (SEQ ID NO: 39), QLSFEECMMLMGK (SEQ ID NO: 40), ELSFEEYIVLVAK (SEQ ID NO: 41), QLSFEEFVILMAR (SEQ ID NO: 42), QLNFEEFSILVGR (SEQ ID NO: 43), and QVDFKEFSMMMAR (SEQ ID NO: 44).

3. An immunoglobulin or proteinaceous binding partner having a binding specificity to an epitope of a vertebrate S100A8 protein, wherein the epitope has an amino acid sequence of a region corresponding to the amino acid sequence ranging from amino acid position 55 to amino acid position 71 of the human protein S100A8 of Uniprot/Swissprot accession number P05109 (SEQ ID NO: 78).

4. The immunoglobulin or proteinaceous binding partner of claim 3, wherein the amino acid sequence is one of the sequences FKELDINTDGAVNFQEF (SEQ ID NO: 5), FKELDINTDGAINFQEF (SEQ ID NO: 45), FKELDINSDGAINFQEF (SEQ ID NO: 46), FKELDINEDGAVNFQEF (SEQ ID NO: 47), FKELDINKDGAVNFEEF (SEQ ID NO: 48), FKELDINSDGASNFQEF (SEQ ID NO: 49), FKELDVNSDGAINFEEF (SEQ ID NO: 50), FKQFDINEDGAVNFQEF (SEQ ID NO: 51), FRQLDINEDGAVNFQEF (SEQ ID NO: 52), FKELDINQDNAVNFEEF (SEQ ID NO: 53), FNELDINSDNAINFQEF (SEQ ID NO: 54), FKELDINQDGGINFEEF (SEQ ID NO: 55), FKELDVNSDSAINFEEF (SEQ ID NO: 56), FKELDVNSDNAINFEEF (SEQ ID NO: 57), FQELDVNSDGAINFEEF (SEQ ID NO: 58), FRELDINSDNAINFEEF (SEQ ID NO: 59), FKELDFTADGAINFEEF (SEQ ID NO: 60), FKELDINQDG GINLEEF (SEQ ID NO: 61), FKELDINQDGFINFEEF (SEQ ID NO: 62), and FKELDSNKDQQINFEEF (SEQ ID NO: 63).

5. (canceled)

6. The method of claim 11, wherein the condition is selected from rheumatoid arthritis, juvenile idiopathic arthritis, psoriatic arthritis, immune reconstituation inflammatory syndrome (IRIS), sepsis, systemic inflammatory response syndrome (SIRS), pneumonia, osteomyelitis, autoinflammatory syndromes, hyperzincemia, systemic inflammation, atherosclerosis, acute coronary syndrome, myocardial infarction, diabetes, an inflammatory skin disease, psoriasis, inflammatory bowel disease, vasculitis, allograft rejection, glomerulonephritis, systemic lupus erythematosus, pancreatitis, a cancer, dermatomyositis and polymyositis, multiple sclerosis, allergies, infections, pulmonary inflammation, acute lung injury (ALI) and its most severe form, acute respiratory distress syndrome (ARDS).

7. A combination of one or more immunoglobulins or proteinaceous binding partners of claim 1 and the immunoglobulin or proteinaceous binding partner having a binding specificity to an epitope of a vertebrate S100A8 protein, wherein the epitope has an amino acid sequence of a region corresponding to the amino acid sequence ranging from amino acid position 55 to amino acid position 71 of the human protein S100A8 of Uniprot/Swissprot accession number P05109 (SEQ ID NO: 78).

8. The combination of claim 7, being comprised in a single immunoglobulin or proteinaceous binding partner, the immunoglobulin or proteinaceous binding partner having at least a dual binding specificity.

9. (canceled)

10. The method of claim 54, wherein the condition is selected from rheumatoid arthritis, juvenile idiopathic arthritis, psoriatic arthritis, immune reconstituation inflammatory syndrome (IRIS), sepsis, systemic inflammatory response syndrome (SIRS), pneumonia, osteomyelitis, autoinflammatory syndromes, hyperzincemia, systemic inflammation, atherosclerosis, acute coronary syndrome, myocardial infarction, diabetes, an inflammatory skin disease, psoriasis, inflammatory bowel disease, vasculitis, allograft rejection, glomerulonephritis, systemic lupus erythematosus, pancreatitis, a cancer, dermatomyositis and polymyositis, multiple sclerosis, allergies, infections, pulmonary inflammation, acute lung injury (ALI) and its most severe form, acute respiratory distress syndrome (ARDS).

11. A method of treating a subject suffering from an inflammatory condition, the method comprising administering to the subject at least one of the immunoglobulin or proteinaceous binding partner of claim 1.

12. The method of claim 11, wherein the subject is a mammal.

13. An isolated peptide or peptidomimetic comprising the sequence of X₃EX₂X₃X₁X₁X₁ X₁X₁X₁ X₅X₁X₁X₆X₂X₁X₁ (SEQ ID NO: 6), wherein X₁ represents any amino acid, X₂ represents an amino acid with a side chain carrying a carboxylic acid group, X₃ represents a non-polar amino acid, X₅ represents D, N, E or Q, X₆ represents an aromatic amino acid, wherein the peptide differs from a calcium binding protein.

14. The isolated peptide or peptidomimetic of claim 13, wherein the sequence of SEQ ID NO: 6 is (a) the sequence of MEX₂X₃DX₁NX₁DX₁ QX₁X₁FEX₂X₁ (SEQ ID NO: 7), or a homolog thereof; or (b) the sequence of MEDX₃DX₃NX₁DX₁ QX₃X₁FEEX₁ (SEQ ID NO: 8), or a homolog thereof.

15. The isolated peptide or peptidomimetic of claim 13, essentially consisting of the sequence of SEQ ID NO: 6.

16. An isolated peptide or peptidomimetic comprising the sequence of X₅X₁X₁X₆X₂X₁X₁ X₁X₃X₃ X₃X₃X₁ (SEQ ID NO: 9), wherein X₁ represents any amino acid, X₂ represents an amino acid with a side chain carrying a carboxylic acid group, X₃ represents a non-polar amino acid, X₅ represents D, N, E or Q and X₆ represents an aromatic amino acid, wherein the peptide differs from a calcium binding protein.

17. The isolated peptide or peptidomimetic of claim 16, (a) wherein the sequence of SEQ ID NO: 6 is the sequence of QX₁X₁FEX₂X₁X₁X₃X₃X₃X₃X₇ (SEQ ID NO: 10), or a homolog thereof, wherein X₇ represents R or K, or (b) wherein the sequence of SEQ ID NO: 6 is the sequence of QX₃X₁FEEX₁X₁MLMX₃X₇ (SEQ ID NO: 11), or a homolog thereof or (c) essentially consisting of the sequence of SEQ ID NO: 9.

18. An isolated peptide or peptidomimetic comprising the sequence of X₆X₈X₅X₃X₁X₁X₁X₁X₁X₁ X₁X₁NX₃X₅X₁X₆ (SEQ ID NO: 12), or a homolog thereof, wherein X₁ represents any amino acid, X₃ represents a non-polar amino acid, X₅ represents D, N, E or Q, X₆ represents an aromatic amino acid, X₈ represents a polar amino acid, wherein the peptide differs from a calcium binding protein.

19. The isolated peptide or peptidomimetic of claim 18, wherein the sequence of SEQ ID NO: 6 is the sequence of FX₈EX₃DX₁NX₁DX₉X₁X₁₀NX₁₁X₅EF (SEQ ID NO: 13), wherein X₉ represents a polar amino acid or G, wherein X₁₀ represents I, V, S or L, X₁₁ represents F or L, or a homolog thereof.

20. An isolated peptide or peptidomimetic comprising the sequence of SEQ ID NO: 5 or a homolog thereof, wherein the peptide differs from a calcium binding protein.

21. The isolated peptide or peptidomimetic of claim 20, essentially consisting of the sequence of SEQ ID NO: 1 or the homolog thereof.

22. A combination of an isolated peptide or peptidomimetic of claim 13 or an isolated peptide or peptidomimetic comprising the sequence of X₅X₁X₁X₆X₂X₁X₁ X₁X₃X₃ X₃X₃X₁ (SEQ ID NO: 9), wherein X₁ represents any amino acid, X₂ represents an amino acid with a side chain carrying a carboxylic acid group, X₃ represents a non-polar amino acid, X₅ represents D, N, E or Q and X₆ represents an aromatic amino acid, wherein the peptide differs from a calcium binding protein; and
an isolated peptide or peptidomimetic comprising the sequence of X₆X₈X₅X₃X₁X₁X₁X₁X₁X₁ X₁X₁NX₃X₅X₁X₆ (SEQ ID NO: 12), or a homolog thereof, wherein X₁ represents any amino acid, X₃ represents a non-polar amino acid, X₅ represents D, N, E or Q, X₆ represents an aromatic amino acid, X₈ represents a polar amino acid, wherein the peptide differs from a calcium binding protein,
wherein the peptidomimetic comprising the sequence of SEQ ID NO: 6 or 9, and the peptidomimetic comprising the sequence of SEQ ID NO: 12 are comprised in a single chain.

23. The combination of claim 22, wherein the peptide comprising the sequence of SEQ ID NO: 6 or the sequence of SEQ ID NO: 9, and the peptide comprising the sequence of SEQ ID NO: 12, or the homolog thereof, are comprised in a single peptide chain.

24. An isolated nucleic acid molecule comprising one of (a) a sequence encoding a peptide of SEQ ID NO: 6, (b) a sequence encoding a peptide of SEQ ID NO: 9, and (c) a sequence encoding a peptide of SEQ ID NO: 12, or a homolog thereof, wherein the encoded peptide differs from the full-length sequence a calcium binding protein.

25. The isolated nucleic acid molecule of claim 24, essentially consisting of one of the sequence of SEQ ID NO: 6, the sequence encoding a peptide of SEQ ID NO: 9 and the sequence encoding a peptide of SEQ ID NO: 12, or the homolog thereof, and optionally an expression cassette.

26. The isolated nucleic acid molecule of claim 24, being comprised in a vector.

27. An in-vitro method of identifying a compound capable of decreasing or inhibiting the formation of a complex between a peptide comprising one of (i) the amino acid sequence of SEQ ID NO: 6 or 9 and (ii) the amino acid sequence of SEQ ID NO: 12 and a TLR4 receptor or a functional fragment thereof, the functional fragment of the TLR4 receptor comprising the binding site for SEQ ID NO: 1 and SEQ ID NO: 3, respectively, the method comprising
(a) allowing the peptide, the TLR4 receptor, or the functional fragment thereof, and a compound suspected to affect the said complex formation to contact each other, and
(b) detecting the formation of a complex between the peptide and the TLR4 receptor, or the functional fragment thereof.

28. The method of claim 27, wherein the peptide comprising the amino acid sequence of SEQ ID NO: 6 or 9 is a S100A9 protein and/or the peptide comprising the amino acid sequence of SEQ ID NO: 12 is a S100A8 protein.

29. An in-vitro method of identifying a compound capable of increasing the stability of a complex between a S100A8 protein, or a functional fragment thereof, and a S100A9 protein, or functional fragments thereof, the method comprising
(a) allowing the S100A8 protein, or the functional fragment thereof, the S100A9 protein, or the functional fragment thereof, and a compound suspected to affect the said complex formation to contact each other, and
(b) detecting the formation of a complex between the S100A8 protein, or the functional fragment thereof, and the S100A9 protein, or the functional fragment thereof.

30. The method of claim 29, wherein the functional fragment of the S100A8 protein and/or the functional fragment of the S100A9 protein comprises at least one of EF hand I and EF hand II.

31. The method of claim 29, wherein the S100A8 protein, or the functional fragment thereof, the S100A9 protein, or the functional fragment thereof, and the compound suspected to affect the said complex formation are allowed to contact each other in the presence of a salt of calcium, zinc or copper.

32. The method of claim 29, wherein the formation of a heterotetrameric complex between the S100A8 protein, or the functional fragment thereof, and the S100A9 protein, or the functional fragment thereof is detected, and wherein the method is a method of identifying a compound capable of increasing the stability of a heterotetrameric complex between a S100A8 protein, or a functional fragment thereof, and a S100A9 protein, or functional fragments thereof.

33. The method of claim 27, further comprising comparing the formation of the complex to a control measurement.

34. The method of claim 33, wherein the control measurement comprises detecting the formation of the complex between the protein S100A8, or the functional fragment thereof, and the protein S100A9, or the functional fragment thereof, in the absence of a compound suspected to affect the complex formation.

35. (canceled)

36. (canceled)

37. An in-vitro method of diagnosing the risk of occurrence, or the presence, of a condition associated with an inflammation in a subject, the method comprising detecting the amount of a complex between a S100A8 protein and a S100A9 protein in a sample from the subject, wherein a decreased amount of the complex relative to a threshold value, indicates an elevated risk of occurrence, or the presence, of a condition associated with an inflammation.

38. The method of claim 37, comprising contacting the sample with an immunoglobulin or proteinaceous binding partner having a binding specificity to (a) a region of a S100A9 protein that differs from the region toward which the immunoglobulin or proteinaceous binding partner according to claim 1 has a binding specificity, or (b) a region of a S100A8 protein that differs from the region toward which the immunoglobulin or proteinaceous binding partner according to claim 3 has a binding specificity, under non-denaturating conditions, and detecting the amount of the complex between protein S100A8 and the protein S100A9 bound, wherein an increased amount of S100A8 or S100A9 detected by binding to the respective immunoglobulin or proteinaceous binding partner, relative to a threshold value, indicates a decreased amount of a complex between a S100A8 protein and a S100A9 protein.

39. The method of claim 38, wherein detecting the amount of the complex between protein S100A8 and the protein S100A9 bound comprises one of immunoprecipitation, flow cytometry and mass spectrometry.

40. The method of claim 37, comprising contacting the sample with an immunoglobulin or proteinaceous binding partner according to claim 1 or according to claim 3 under non-denaturating conditions and detecting the amount of the S100A8 protein or the S100A9 protein, respectively, bound, wherein an increased amount of the S100A8 protein or the S100A9 protein detected, relative to a threshold value, indicates a decreased amount of a complex between a S100A8 protein and a S100A9 protein.

41. The method of claim 40, wherein the immunoglobulin or proteinaceous binding partner has a binding specificity to a peptide of the species to which the subject belongs.

42. The method of claim 41, wherein the immunoglobulin or proteinaceous binding partner has a binding specificity to a human peptide and wherein the subject is a human.

43. The method of claim 37, further comprising comparing the amount of the complex to a control measurement.

44. The method of claim 43, wherein the control measurement comprises detecting the amount of the complex between the S100A8 protein and the S100A9 protein in a sample from a subject known not to suffer from an inflammatory disorder.

45. The method of claim 37, comprising
(a) contacting a first sample from the subject with an immunoglobulin or proteinaceous binding partner having a binding specificity to (i) a region of a S100A9 protein that differs from the region toward which the immunoglobulin or proteinaceous binding partner according to claim 1 has a binding specificity, or (ii) a region of a S100A8 protein that differs from the region toward which the immunoglobulin or proteinaceous binding partner according to claim 3 has a binding specificity under non-denaturating conditions,
(b) contacting a second sample from the subject with an immunoglobulin or proteinaceous binding partner (i) according to claim 1 or (ii) according to claim 3 under non-denaturating conditions,
(c) detecting the amount of the protein S100A8 or the S100A9 protein, respectively, in the first sample and in the second sample, and
(d) comparing the difference between the S100A8 protein or the S100A9 protein bound in the first sample and in the second sample to a threshold value,
wherein a decreased difference between the protein bound in the first sample and in the second sample, relative to a threshold value, indicates an elevated risk of occurrence, or the presence, of a condition associated with an inflammation.

46. The method of claim 45, wherein the threshold value is based on the formation of a corresponding complex to a control measurement.

47. The method of claim 46, wherein the control measurement comprises determining the difference in the amount of the S100A8 protein or the S100A9 protein in a third and a fourth sample, the third and a fourth sample being from a subject known not to suffer from an inflammatory disorder.

48. The method of claim 45, wherein
(a) the immunoglobulin or proteinaceous binding partner contacted with the first sample has a binding specificity to a region of a S100A9 protein that differs from the region toward which the immunoglobulin or proteinaceous binding partner according to claim 1 has a binding specificity, and the immunoglobulin or proteinaceous binding partner contacted with the second sample is an immunoglobulin or proteinaceous binding partner according to claim 1, or
(b) the immunoglobulin or proteinaceous binding partner contacted with the first sample has a binding specificity to a region of a S100A9 protein that differs from the region toward which the immunoglobulin or proteinaceous binding partner according to claim 3 has a binding specificity and the immunoglobulin or proteinaceous binding partner contacted with the second sample is an immunoglobulin or proteinaceous binding partner according to claim 3.

49. The method of claim 37, wherein the sample is one of a blood sample, a plasma sample and a serum sample.

50. A method of treating a subject suffering from an inflammatory disorder, the method comprising administering to the subject a compound obtained by the method of claim 29, thereby increasing the stability of a complex between a S100A8 protein and a S100A9 protein in a body fluid of the subject.

51. A method of treating a subject suffering from an inflammatory disorder, the method comprising administering to the subject a compound obtained by the method of claim 27, thereby decreasing or inhibiting the formation of a complex between the protein S100A8 or the protein S100A9 and a TLR4 receptor on cells of the subject.

52. A method of identifying a binding partner of the isolated peptide or peptidomimetic of claim 13, in an organism, the method comprising
(a) contacting the isolated peptide or peptidomimetic with a sample from the organism, thereby forming a reaction mixture,
(b) allowing a complex to form between the isolated peptide or peptidomimetic and a binding partner in the reaction mixture,
(c) isolating the peptide or peptidomimetic from the reaction mixture, wherein the peptide or peptidomimetic is comprised in a complex with the binding partner, and
(d) analysing the binding partner.

53. The method of claim 52, wherein isolating the peptide or peptidomimetic from the reaction mixture comprises one of immunoprecipitation, chromatography and flow cytometry.

54. The method of claim 11 comprising administering to the subject an immunoglobulin or proteinaceous binding partner having a binding specificity to an epitope of a vertebrate S100A8 protein, wherein the epitope has an amino acid sequence of a region corresponding to the amino acid sequence ranging from amino acid position 55 to amino acid position 71 of the human protein S100A8 of Uniprot/Swissprot accession number P05109 (SEQ ID NO: 78).


SEQUENCE LISTING (87 sequences)

SEQ ID NO: 1 - protein, 17 aa, Artificial Sequence
  synthetic peptide
MEDLDTNADKQLSFEEF

SEQ ID NO: 2 - protein, 17 aa, Artificial Sequence
  synthetic peptide
FXXXXXXXXXXXNXXXF

SEQ ID NO: 3 - protein, 16 aa, Artificial Sequence
  synthetic peptide
QLSFEEFIMLMARTTT

SEQ ID NO: 4 - protein, 17 aa, Artificial Sequence
  synthetic peptide
FXXXXXXXXXXXNXXXF

SEQ ID NO: 5 - protein, 17 aa, Artificial Sequence
  synthetic peptide
FKELDINTDGAVNFQEF

SEQ ID NO: 6 - protein, 17 aa, Artificial Sequence
  synthetic peptide
XEXXXXXXXXXXXXXXX

SEQ ID NO: 7 - protein, 17 aa, Artificial Sequence
  synthetic peptide
MEXXDXNXDXQXXFEXX

SEQ ID NO: 8 - protein, 17 aa, Artificial Sequence
  synthetic peptide
MEDXDXNXDXQXXFEEX

SEQ ID NO: 9 - protein, 13 aa, Artificial Sequence
  synthetic peptide
XXXXXXXXXXXXX

SEQ ID NO: 10 - protein, 13 aa, Artificial Sequence
  synthetic peptide
QXXFEXXXXXXXX

SEQ ID NO: 11 - protein, 13 aa, Artificial Sequence
  synthetic peptide
QXXFEEXXMLMXX

SEQ ID NO: 12 - protein, 17 aa, Artificial Sequence
  synthetic peptide
XXXXXXXXXXXXNXXXX

SEQ ID NO: 13 - protein, 17 aa, Artificial Sequence
  synthetic peptide
FXEXDXNXDXXXNXXEF

SEQ ID NO: 14 - protein, 17 aa, Artificial Sequence
  synthetic peptide
MEDLDTNEDKQLSFEEF

SEQ ID NO: 15 - protein, 17 aa, Artificial Sequence
  synthetic peptide
MEDLDTNVDKQLSFEEF

SEQ ID NO: 16 - protein, 17 aa, Artificial Sequence
  synthetic peptide
MEDLDTNLDKQLSFEEF

SEQ ID NO: 17 - protein, 17 aa, Artificial Sequence
  synthetic peptide
MEDLDTNGDKQLNFEEF

SEQ ID NO: 18 - protein, 17 aa, Artificial Sequence
  synthetic peptide
LEDLDTNADKQLTFEEF

SEQ ID NO: 19 - protein, 17 aa, Artificial Sequence
  synthetic peptide
LEDLDTNVDKQLSFEEF

SEQ ID NO: 20 - protein, 17 aa, Artificial Sequence
  synthetic peptide
LEDLDTNEDKQLSFEEF

SEQ ID NO: 21 - protein, 17 aa, Artificial Sequence
  synthetic peptide
MEDLDTNGDKELNFEEF

SEQ ID NO: 22 - protein, 17 aa, Artificial Sequence
  synthetic peptide
MEDLDTNEDKELSFEEY

SEQ ID NO: 23 - protein, 17 aa, Artificial Sequence
  synthetic peptide
LEDLDTNGDKQLNFEEF

SEQ ID NO: 24 - protein, 17 aa, Artificial Sequence
  synthetic peptide
MEDLDTNQDNQLSFEEC

SEQ ID NO: 25 - protein, 17 aa, Artificial Sequence
  synthetic peptide
MEDLDTNLDQQLSFEEL

SEQ ID NO: 26 - protein, 17 aa, Artificial Sequence
  synthetic peptide
MQDLDTNQDQQLSFEEV

SEQ ID NO: 27 - protein, 17 aa, Artificial Sequence
  synthetic peptide
MEDLDTNQDKQLSFEEF

SEQ ID NO: 28 - protein, 17 aa, Artificial Sequence
  synthetic peptide
MQELDTNQNGQVDFKEF

SEQ ID NO: 29 - protein, 17 aa, Artificial Sequence
  synthetic peptide
FEETDLNKDKELTFEEF

SEQ ID NO: 30 - protein, 13 aa, Artificial Sequence
  synthetic peptide
QLSFEEFIVLMAR

SEQ ID NO: 31 - protein, 13 aa, Artificial Sequence
  synthetic peptide
QLSFEEFIMLVAR

SEQ ID NO: 32 - protein, 13 aa, Artificial Sequence
  synthetic peptide
QLTFEEFIMLMGR

SEQ ID NO: 33 - protein, 13 aa, Artificial Sequence
  synthetic peptide
QLSFEEFIMLVIR

SEQ ID NO: 34 - protein, 13 aa, Artificial Sequence
  synthetic peptide
QLSFEEFIILVAR

SEQ ID NO: 35 - protein, 13 aa, Artificial Sequence
  synthetic peptide
QLSFEELTMLLAR

SEQ ID NO: 36 - protein, 13 aa, Artificial Sequence
  synthetic peptide
QLSFEEVIMLFAR

SEQ ID NO: 37 - protein, 13 aa, Artificial Sequence
  synthetic peptide
QLSFEEFSILMAK

SEQ ID NO: 38 - protein, 13 aa, Artificial Sequence
  synthetic peptide
QLSFEEFSMLVAK

SEQ ID NO: 39 - protein, 13 aa, Artificial Sequence
  synthetic peptide
QLSFEECMMLMAK

SEQ ID NO: 40 - protein, 13 aa, Artificial Sequence
  synthetic peptide
QLSFEECMMLMGK

SEQ ID NO: 41 - protein, 13 aa, Artificial Sequence
  synthetic peptide
ELSFEEYIVLVAK

SEQ ID NO: 42 - protein, 13 aa, Artificial Sequence
  synthetic peptide
QLSFEEFVILMAR

SEQ ID NO: 43 - protein, 13 aa, Artificial Sequence
  synthetic peptide
QLNFEEFSILVGR

SEQ ID NO: 44 - protein, 13 aa, Artificial Sequence
  synthetic peptide
QVDFKEFSMMMAR

SEQ ID NO: 45 - protein, 17 aa, Artificial Sequence
  synthetic peptide
FKELDINTDGAINFQEF

SEQ ID NO: 46 - protein, 17 aa, Artificial Sequence
  synthetic peptide
FKELDINSDGAINFQEF

SEQ ID NO: 47 - protein, 17 aa, Artificial Sequence
  synthetic peptide
FKELDINEDGAVNFQEF

SEQ ID NO: 48 - protein, 17 aa, Artificial Sequence
  synthetic peptide
FKELDINKDGAVNFEEF

SEQ ID NO: 49 - protein, 17 aa, Artificial Sequence
  synthetic peptide
FKELDINSDGASNFQEF

SEQ ID NO: 50 - protein, 17 aa, Artificial Sequence
  synthetic peptide
FKELDVNSDGAINFEEF

SEQ ID NO: 51 - protein, 17 aa, Artificial Sequence
  synthetic peptide
FKQFDINEDGAVNFQEF

SEQ ID NO: 52 - protein, 17 aa, Artificial Sequence
  synthetic peptide
FRQLDINEDGAVNFQEF

SEQ ID NO: 53 - protein, 17 aa, Artificial Sequence
  synthetic peptide
FKELDINQDNAVNFEEF

SEQ ID NO: 54 - protein, 17 aa, Artificial Sequence
  synthetic peptide
FNELDINSDNAINFQEF

SEQ ID NO: 55 - protein, 17 aa, Artificial Sequence
  synthetic peptide
FKELDINQDGGINFEEF

SEQ ID NO: 56 - protein, 17 aa, Artificial Sequence
  synthetic peptide
FKELDVNSDSAINFEEF

SEQ ID NO: 57 - protein, 17 aa, Artificial Sequence
  synthetic peptide
FKELDVNSDNAINFEEF

SEQ ID NO: 58 - protein, 17 aa, Artificial Sequence
  synthetic peptide
FQELDVNSDGAINFEEF

SEQ ID NO: 59 - protein, 17 aa, Artificial Sequence
  synthetic peptide
FRELDINSDNAINFEEF

SEQ ID NO: 60 - protein, 17 aa, Artificial Sequence
  synthetic peptide
FKELDFTADGAINFEEF

SEQ ID NO: 61 - protein, 17 aa, Artificial Sequence
  synthetic peptide
FKELDINQDGGINLEEF

SEQ ID NO: 62 - protein, 17 aa, Artificial Sequence
  synthetic peptide
FKELDINQDGFINFEEF

SEQ ID NO: 63 - protein, 17 aa, Artificial Sequence
  synthetic peptide
FKELDSNKDQQINFEEF

SEQ ID NO: 64 - protein, 17 aa, Artificial Sequence
  synthetic peptide
XEXXXXXXXXQXXXEXX

SEQ ID NO: 65 - protein, 13 aa, Artificial Sequence
  synthetic peptide
QXXXEXXXXXXXX

SEQ ID NO: 66 - protein, 17 aa, Artificial Sequence
  synthetic peptide
XEXXXXXXXXXXXXXXX

SEQ ID NO: 67 - protein, 17 aa, Artificial Sequence
  synthetic peptide
MEXXXXNXXXQXXFEXX

SEQ ID NO: 68 - protein, 13 aa, Artificial Sequence
  synthetic peptide
XXXXXXXXXXXXX

SEQ ID NO: 69 - protein, 17 aa, Artificial Sequence
  synthetic peptide
FXXXXXXXDXXXNXXXF

SEQ ID NO: 70 - protein, 17 aa, Artificial Sequence
  synthetic peptide
FXXXXXXXXXXXNXXEF

SEQ ID NO: 71 - protein, 17 aa, Artificial Sequence
  synthetic peptide
FXXXXXXXXXXXNXXEF

SEQ ID NO: 72 - protein, 17 aa, Artificial Sequence
  synthetic peptide
MEDXXXXXDXQXXFEXX

SEQ ID NO: 73 - protein, 17 aa, Artificial Sequence
  synthetic peptide
MEDXXXXXXXQXXFEXX

SEQ ID NO: 74 - protein, 17 aa, Artificial Sequence
  synthetic peptide
MEXXXXXXXXQXXFEXX

SEQ ID NO: 75 - protein, 17 aa, Artificial Sequence
  synthetic peptide
MEXXXXXXXXQXXFEXX

SEQ ID NO: 76 - protein, 17 aa, Artificial Sequence
  synthetic peptide
MEXXXXXXXXQXXFEXX

SEQ ID NO: 77 - protein, 114 aa, Homo sapiens
MTCKMSQLERNIETIINTFHQYSVKLGHPDTLNQGEFKELVRKDLQNFLKKENKNEKVIEHIMEDLDTNADKQLSFEEFIMLMARLTWASHEKMHEGDEGPGHHHKPGLGEGTP

SEQ ID NO: 78 - protein, 93 aa, Homo sapiens
MLTELEKALNSIIDVYHKYSLIKGNFHAVYRDDLKKLLETECPQYIRKKGADVWFKELDINTDGAVNFQEFLILVIKMGVAAHKKSHEESHKE

SEQ ID NO: 79 - protein, 131 aa, Equus caballus
MAELSQMERDIETIINVFHQYSVRLGHPDTLNRKEFKQLVQKELANFLKSKKKDEKAINHIMEDLDTNEDKQLSFEEFIILVARLTHASHEKMHEHDQGHGHCHGPGLGESGHGHSHGGHGHSHGGHGHSH

SEQ ID NO: 80 - protein, 113 aa, Callithrix jacchus
AFEMSQLESSIETIINTFHHYSVRLGHPDALNQKEFKDLVQKELQNFLKKEKRNEQDINHILEDLDTNADKQLTFEEFIMLMGRLTWASHEHMHKNDHGPGHCHGPGLGEGTP

SEQ ID NO: 81 - protein, 113 aa, Callithrix jacchus; identical to SEQ ID NO: 80

SEQ ID NO: 82 - protein, 91 aa, Monodelphis domestica
MATKLECAINCLVEVFHKYSLTGGHPHALSREQFGKLLEKECSEFTKKSKKTVPEFMKELDINQDGFINFEEFLILTLKMVIEHHEDSHKE

SEQ ID NO: 83 - protein, 113 aa, Mus musculus
MANKAPSQMERSITTIIDTFHQYSRKEGHPDTLSKKEFRQMVEAQLATFMKKEKRNEALINDIMEDLDTNQDNQLSFEECMMLMAKLIFACHEKLHENNPRGHGHSHGKGCGK

SEQ ID NO: 84 - protein, 113 aa, Rattus norvegicus
MAAKTGSQLERSISTIINVFHQYSRKYGHPDTLNKAEFKEMVNKDLPNFLKREKRNENLLRDIMEDLDTNQDNQLSFEECMMLMGKLIFACHEKLHENNPRGHDHSHGKGCGK

SEQ ID NO: 85 - protein, 147 aa, Bos Taurus
MEDKMSQMESSIETIINIFHQYSVRLGHYDTLIQKEFKQLVQKELPNFLKKQKKNEAAINEIMEDLDTNVDKQLSFEEFIMLVARLTVASHEEMHNTAPPGPGHRHGPGYGKGSPDQGSHDQGSHGHGHGHSHGGHGHSHGGHGHSH

SEQ ID NO: 86 - protein, 152 aa, Myotis davidii
MSQTMMECSVETIINIFHQYSTRLGHPDRLNQKEFSQMVKKELPNFLKKEKRNEALIRDILEDLDTSGDKDLSFEEFITLVGRLTEASHEEMHKNTPKGHGHAHGPGFGGSGRNPCKTQGGNQGGGHGHSHDGHGHSHDGHGHSHDGHGHSH

SEQ ID NO: 87 - protein, 188 aa, Mustela putorius furo
PFLALTVQLRVATLLTCEAIFARELRFWPGAAYKCWAHTAPSHPSVWLWDLDRVQEYRKMADQMSQLECSIETIINIFHQYSVRMEHTDMLNQKELKQLVKKELPNFLKKQKKNDNTINKIMEDLDTNGDKQLNFEEFSILVGRLTMASHEEMHKNAPEGEGHSHGPGFGQGDQGHCHSHGGHGHGHS